(19) United States
(12) Patent Application Publication
(10) Pub. No.: US 2002/0156247 A1
(43) Pub. Date: Oct. 24, 2002

(54) Mammalian checkpoint genes and proteins

(76) Inventors: Stephen J. Elledge, Houston, TX (US); Yolanda Sanchez, Cincinnati, OH (US)

Correspondence Address: Tim Headley, GARDERE WYNNE SEWELL LLP, Suite 3400, 1000 Louisiana, Houston, TX, 77002-5007, US

(21) Appl. No.: 10/020,038

(22) Filed: Dec. 12, 2001

Related U.S. Application Data
(62) Division of application No. 09/488,364, filed on Jan. 12, 2000, now Pat. No. 6,307,015.

(51) Int. Cl. C07K 16/40

(52) U.S. Cl. 530/389.1

(57) ABSTRACT

The present invention relates to the isolation of gene sequences encoding mammalian cell cycle checkpoints, as well as the expression of the encoded proteins using recombinant DNA technology. The expressed proteins are used to generate specific antibodies and to inhibit the growth of cells. The human checkpoint gene sequences are used as a probe for a portion of the chromosome associated with tumors and other malignancies, as well as growth and/or development deficiencies.

This Application is a Division Application of co-pending U.S. patent application Ser. No., 09/488,364, filed Jan. 12, 2001, entitled “Mammalian Check Genes and Proteins,” now U.S. Pat. No. 6,307,015, which claims priority to U.S. patent application Ser. No. 08/924,183 filed on Sep. 5, 1997, entitled, “Mammalian Checkpoint Genes And Proteins,” now U.S. Pat. No., 6,218,109 B1.

### FIELD OF THE INVENTION

The present invention relates to mammalian proteins and gene sequences involved in cellular responses to DNA damage. In particular, the present invention provides checkpoint genes and proteins.

### BACKGROUND OF THE INVENTION

The proper development of a multicellular organism is a complex process that requires precise spatial and temporal control of cell proliferation. Cell proliferation in the embryo is controlled via an intricate network of extracellular and intracellular signaling pathways that process growth regulatory signals. This signaling network is superimposed upon the basic cell cycle regulatory machinery that controls particular cell cycle transitions.

Cell cycle checkpoints are regulatory pathways that control the order and timing of cell cycle transitions, and ensure that critical events such as DNA replication and chromosome segregation are completed with high fidelity. For example, proliferating eukaryotic cells arrest their progression through the cell cycle in response to DNA damage. This arrest is critical to the survival of the organism, as failure to repair damaged DNA can result in the formation and transfer of mutations, damaged chromosomes, cancer, or other detrimental effects. The mechanism responsible for monitoring the integrity of the organism's DNA and preventing the progression through the cell cycle when DNA damage is detected is referred to as the “DNA damage checkpoint.”

In response to DNA damage, cells activate a checkpoint pathway that arrests the cell cycle, in order to provide time for repair, and induces the transcription of genes that facilitate the needed repair. In yeast, this checkpoint pathway consists of several protein kinases including phosphoinositide (PI)-kinase homologs HATM (human), scmec I (Saccharomyces cerevisiae), spRad3 (Schizosaccharomyces pombe), and protein kinases scdunl (Saccharomyces cerevisiae), scRad53 (Saccharomyces cerevisiae), and spchkl (Schizosaccharomyces pombe) (See e.g., S. Elledge, Science 1664 [1996]).

Indeed, the ability to coordinate cell cycle transitions in response to genotoxic and other stressors is critical to the maintenance of genetic stability and prevention of uncontrolled cellular growth. Loss of a checkpoint gene leads to genetic instability and the inability of the cells to deal with genomic insults such as those suffered as a result of the daily exposure to ultraviolet radiation. The loss of negative growth control and improper monitoring of the fidelity of DNA replication are common features of tumor cells. When checkpoints are eliminated (e.g., by mutation or other means), cell death, infidelity in chromosome transmission, and/or increased susceptibility to deleterious environmental factors (e.g., DNA-damaging agents) result. A variety of abnormal cells arising due to infidelity during mitoses have been detected in humans, including aneuploidy, gene amplification, and multipolar mitoses (See, L. H. Hartwell and T. A. Weinert, Science 246:629 [1989]).

Accordingly, elucidation of checkpoint function, as well as the disruption of checkpoint function, will further the understanding of the process of cellular transformation (i.e., the conversion of normal cells to a state of unregulated growth), as well as cell differentiation and organismal development.

### SUMMARY OF THE INVENTION

The present invention provides mammalian proteins and gene sequences involved in cellular responses to DNA damage. In particular, the present invention provides Chkl genes and proteins.

In one embodiment, the present invention provides the nucleotide sequence set forth in SEQ ID NO: 1. In alternative embodiments, the present invention provides SEQ ID NO: 1, wherein it further comprises 5′ and 3′ flanking regions. In yet another embodiment, the sequence further comprises intervening regions. In a further embodiment, the present invention also provides a polynucleotide sequence which is complementary to SEQ ID NO: 1 or variants thereof In a preferred embodiment, the present invention provides a vector comprising the nucleotide sequence of claim 1 The present invention also provides host cell(s) containing the vector of claim 4.

The present invention also provides a purified Chkl protein encoded by the nucleotide sequence of claim 1, as well as a purified protein comprising the amino acid sequence set forth in SEQ ID NO: 3. In addition, the present invention provides fusion proteins comprising a least a portion of the human Chkl protein, as well as non-Chkl protein sequences. It is not intended that the fusion proteins of the present invention be limited to any particular portion of the Chkl portion or any particular non-Chkl protein sequences. In preferred embodiments, the fusion the Chkl protein portion of the fusion protein comprises at least a portion of SEQ ID NO: 3. In an alternative embodiment, the non-Chkl protein sequence comprises an affinity tag. In particularly preferred embodiment, the affinity tag comprises a histidine tract.

In yet another embodiment, the present invention provides the sequence set forth in SEQ ID NO: 2. In an alternative embodiment, the nucleotide sequence further comprises 5′ and 3′ flanking regions. In another alternative embodiment, the nucleotide sequence further comprises intervening regions. In yet another embodiment, the present invention provides a polynucleotide sequence that is complementary to SEQ ID NO: 2 or variants thereof.

The present invention also provides a vector comprising the nucleotide sequence set forth in SEQ ID NO: 2. In one preferred embodiment, the present invention provides a host cell containing the vector comprising this nucleotide sequence.

The present invention further provides a purified Chkl protein encoded by the nucleotide sequence of SEQ ID NO: 2. In yet another embodiment, the present invention provides a purified protein comprising the amino acid sequence set forth in SEQ ID NO: 4.

The present invention also provides fusions proteins comprising at least a portion of the murine Chkl protein and a non-Chkl protein sequence. It is not intended that the fusion proteins of the present invention be limited to any particular portion of the Chkl portion or any particular non-Chkl protein sequences. In preferred embodiments, the fusion the Chkl protein portion of the fusion protein comprises at least a portion of SEQ ID NO: 4. In an alternative embodiment, the nonChkl protein sequence comprises an affinity tag. In particularly preferred embodiment, the affinity tag comprises a histidine tract.

The present invention also provides methods for detecting Chkl protein. In one embodiment, the method comprises the steps of providing in any order: a sample suspected of containing the Chkl protein; an antibody capable of specifically binding to a Chkl protein; mixing the sample and the antibody under conditions wherein the antibody can bind to the Chkl protein; and detecting the binding. In one alternative embodiment, the sample comprises one or more cells suspected of containing Chkl protein. In yet another embodiment, the cells contain an abnormal Chkl protein. In a further embodiment, the cells are selected from the group consisting of human cells and murine cells.

The present invention also provides antibodies capable of recognizing at least a portion of human and/or murine Chkl protein. In one embodiment, the present invention provides an antibody, wherein the antibody is capable of specifically binding to at least one antigenic determinant on the proteins encoded by an amino acid sequence selected from the group comprising SEQ ID NOS: 3, 4, 7, 8, 9, and 10. In one preferred embodiment, the antibody is a polyclonal antibody, while in an alternative embodiment, the antibody is a monoclonal antibody.

The present invention also provides methods for producing antibodies comprising the steps of providing in any order: an antigen comprising at least a portion of Chkl protein; and an animal having immunocompetent cells; and exposing the animal to the Chkl protein under conditions such that the immunocompetent cells produce anti-Chkl antibodies. In one alternative embodiment, the method further comprises the step of harvesting the antibodies. In another alternative embodiment, the antigen comprises at least a portion of Chkl protein is a fusion protein. In yet another embodiment, the method further comprises the step of fusing the immunocompetent cells with an immortal cell line under conditions such that an hybridoma is produced.

The present invention also provides methods for detection of polynucleotides encoding human and/or murine Chkl in biological samples. It is not intended that the method be limited to any particular sequence contained within SEQ ID NOS: L or 2. Indeed, it is contemplated that any sequence be used in the method, including degenerate primers that are based on the sequence of Chkl and that are capable of recognizing at least a portion of the Chkl gene.

In one embodiment, the method comprises the steps of hybridizing a nucleotide comprising at least a portion of the nucleotide of SEQ ID NO: 1 to nucleic acid material of a biological sample, thereby forming a hybridization complex; and detecting the hybridization complex, wherein the presence of the complex correlates with the presence of a polynucleotide encoding human Chkl in the biological sample.

In an alternative embodiment of the method, the biological sample is amplified by the polymerase chain reaction before hybridization. In yet another alternative method, polymerase chain reaction is conducted using primers selected from the group consisting of SEQ ID NOS: 5, 6, 12, 13, 14, and 15.

### DESCRIPTION OF THE FIGURES

FIG. 1 shows the cDNA sequence of human Chkl (SEQ ID NO: 1), as well as the predicted amino acid sequence (SEQ ID NO: 3).

FIG. 2 shows the cDNA sequence of murine Chkl (SEQ ID NO: 2), as well as the predicted amino acid sequence (SEQ ID NO: 4).

FIG. 3 shows the domain structure of the predicted human Chkl.

FIG. 4 shows the alignment of human, D. melanogaster, C elegans, and S. pombe Chkl homologs.

FIG. 5A shows the expression pattern of human Chkl mRNA in different adult tissues, as determined by Northern analysis.

FIG. 5B shows the expression pattern of murine Chkl mRNA in different adult tissues, as determined by Northern analysis.

FIG. 6 shows the reactivity of a 54 kD protein from different cell lines, with purifed antibodies directed to a peptide (anti-PEP), or full-length Chkl protein anti-FL).

FIG. 7A shows that Chkl is modified in response to DNA damage in HeLa cells.

FIG. 7B shows that Chkl in modified in response to DNA damage in Jurkat cells.

FIG. 8A is an autoradiograph showing radiolabeled phosphate incorporation due to phosphorylation of Chkl, Cdc25A, Cdc25B, and Cdc25C.

FIG. 8B is a Coomassie stained gel of FIG. 8A, showing the amount of each protein that is present in each lane.

FIG. 9A is an autoradiograph showing radiolabeled phosphate incorporation due to phosphorylation of Chkl and wild-type Cdc25C(200-256).

FIG. 9B is a Coommassie-stained gel of FIG. 9A, showing the amount of each protein present in each lane.

FIG. 9C is an autoradiograph showing radiolabeled phosphate incorporation due to phosphorylation of Chkl and wild-type Cdc25C.

FIG. 9D is a Coomassie-stained gel of FIG. 9C, showing he amount of each protein present in each lane.

FIG. 10 shows Chkl binding to GST-Cdc25A, GST-Cdc25B and GST-Cdc25C by immunoblotting with anti Chkl antibodies.

FIG. 11 shows the proteolytic sites surrounding serine 216 on Cdc25C.

FIG. 12A shows the results of mapping of the phosphorylation site on Cdc25C by Chkl by proteolytic cleavage and sequencing of phosphorylated Cdc25C, as resolved by HPLC.

FIG. 12B provides identification of phosphorylated residue by manual Edman degradation of tryptic peptide present in fraction 57 shown in FIG. 12A.

### DESCRIPTION OF THE INVENTION

The cell cycle comprises a collection of highly ordered processes that lead to the duplication of cells. As cells move through the cell cycle, they undergo several discrete transitions (i.e., an unidirectional change of state in which a cell that was performing one set of processes shifts its activity to perform a different set of processes). Although the mechanism of how these transitions are coordinated to occur at precise times and in a defined order remains unknown, in principle, the ordering of cell cycle events may be accomplished by requiring the next event to physically require the completion of the previous event. This pathway has been referred to as a “substrate-product relationship” (Hartwell and Weinert, Science 246:629 [1989]). However, other research has shown that the predominant mechanism for dependency relies upon positive or negative regulatory circuits.

These regulatory circuits are surveillance mechanisms that monitor the completion of critical cell cycle events, and allow the occurrence of subsequent cell cycle transitions. Two classes of regulatory circuits have been described. “Intrinsic” mechanism act in each cell cycle to order events, while “extrinsic” mechanisms are induced to act only when a defect is detected. Both of these mechanisms may use the same components to enforce cell cycle arrest. These pathways are particularly important, as their loss leads to reduced fidelity of cell cycle events such as chromosome duplication and segregation. Also, such alterations decrease the reproductive fitness of unicellular organisms, and may lead to uncontrolled cellular proliferation and cancer in multi-cellular organisms.

The term “checkpoint” is used to refer to particular subsets of these intrinsic and extrinsic mechanisms. As used herein, a “checkpoint” is a biochemical pathway that ensures dependence of one process upon another process that is otherwise biochemically lo unrelated. As a null allele in a checkpoint gene results in a loss of this dependency, checkpoints are inhibitory pathways. This definition is broad, and can apply to many situations that occur in multicellular organisms, particularly during development. However, it is often used in reference to control of cell cycle transitions. In preferred embodiments, the term refers to the biochemical pathway that ensures dependency. For example, the DNA-damage checkpoint is the mechanism by which damaged DNA is detected and a signal is generated that arrests cells in the GI phase of the cell cycle, slows down S phase (i.e., DNA synthesis), arrests cells in the G2 phase, and induces the transcription of repair genes. The position of arrest within the cell cycle varies, depending upon the phase in which the damage is determined. Whether the loss of a checkpoint has an immediate consequence for an organism during a normal cell cycle depends upon the particular pathway involved and the inherent timing of the processes. Thus, timing and checkpoints can act as redundant controls, in order to ensure the proper order of events. Therefore, there are no constraints on whether checkpoints are essential or inducible (extrinsic).

To address the conservation of checkpoint function, a search for human homologs of yeast checkpoint genes was conducted using a degenerate polymerase chain reaction (PCR) strategy. This search identified a human gene very similar to spChkl (See, FIG. 3). Using hchk I CDNA (SEQ ID NO: 1) as a probe, the mchk I gene from mouse (SEQ ID NO: 2) was isolated. The sequence of the longest human cDNA (1891 hp) predicted a translation product of 476 amino acids with a molecular size of 54 kD (FIG. 3). No in-frame stop codon was found upstream of the first methionine, which is within the Kozak consensus sequence, and is likely to be the bone fide initiation codon because its encoded protein is the same size as that observed in cells (see below). hChkl was found to be related to a C elegans gene in the database and a D. melanogaster gene, grp, with a role in cell cycle control and development (See e.g., FIG. 3). The predicted hChkl is 29% identical and 44% similar to spChkl, 40% identical and 56% similar to the ceChkl, and 44% identical and 56% similar to dmChkl. Sequence analysis revealed several COOH-terminal domains that are highly conserved in the Chkl family of kinases (See e.g., FIG. 4).

The chromosomal location of hChkl was then mapped to 11 q24 by fluorescence in situ hybridization. This site is adjacent to the ATM gene at 11 q23. This was of interest as ATM is mutated in patients with ataxia telangectasia, a fatal disease characterized by autosomal recessive inheritance, immunological impairment, ataxia related to progressive cerebellar Purkinje cell death, and a high incidence of cancer (See e.g., L. S. Cox and D. P. Lane, Bioessays 17:501 [1995]; Y. Shiloh et al., J. Hum. Genet., 3:116 [1995]; K Savitsky et al., Science 268:1749 [1995]); and K. Savitsky et al, Hum. Mol. Genet., 4:2025 [1995]). Approximately 1% of humans are heterozygotic for ATM defects, and show an increased incidence of cancer (See, M. Swift et al., N. Eng. J. Med., 316:1289 [1987]; and M. Swift et al., N. Eng. J. Med., 325:1831 [1991]).

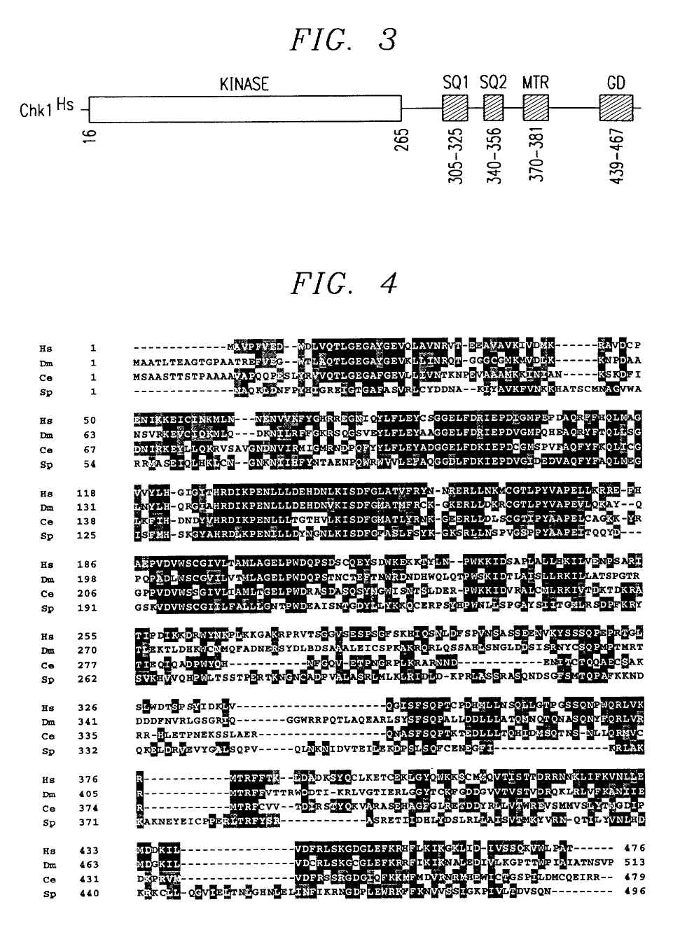

Northern blot analysis revealed ubiquitous expression with large amounts of Chkl expression in human thymus, testis, small intestine and colon (FIG. 5). In this analysis, blots containing the polyadenylated RNA from the indicated tissues were probed with human (FIG. 5A) or mouse (FIG. 5B) chkl cDNAs. As shown in FIG. 5B, in adult mice, mChkl was detected in all tissues examined and in large amounts in the testis, spleen, and lung. In addition, mouse embryos revealed ubiquitous expression, with large amounts detected in the brain, liver, kidney, pancreas, intestines, thymus and lung. This was of interest as testis, spleen and thymus also express large amounts of ATM (G. Chen and E. Y. H. P. Lee, J. Biol. Chem., 271:33693 [1996]; N. D. Lakin et al., Oncogene 13:2707 [1996]).

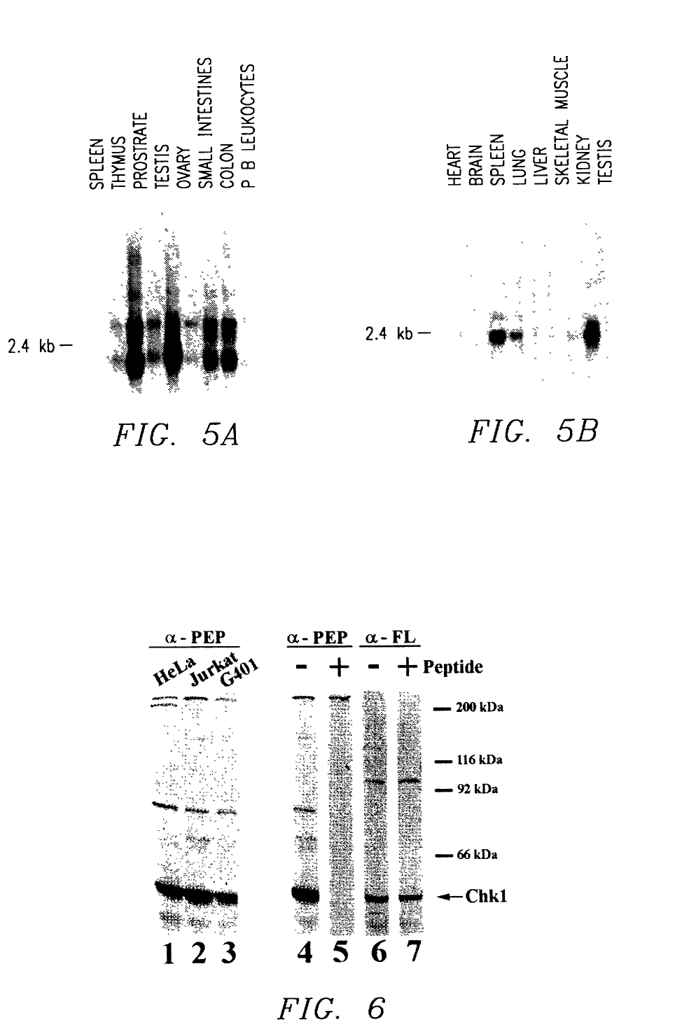

Affinity purified antibodies to hChkl protein (GST fusion hChkl protein) made in baculovirus (anti-FL) or to its COOH-terminal 15 amino acids (anti-PEP) recognized a 54-kD protein (FIG. 6) that comigrates with hChkl expressed in baculovirus. As shown in this Figure, the anti-PEP, but not anti-FL signal is competed by addition of excess peptide, indicating that the two sera are recognizing different hChkl epitopes, further confirming identity of the 54kD band as endogenous hChkl. A 70 kD protein was also specifically recognized by anti-PEP.

When mChkl was expressed from the cytomegalovirus (CMV) promoter in baby hamster kidney (BKH) cells, a 54 kD nuclear protein was detected only in transfected cells using antibodies directed against the C-terminal peptide of mChkl. This exogenous mChkl was found to comigrate with endogenous mChkl from mouse lung tissue.

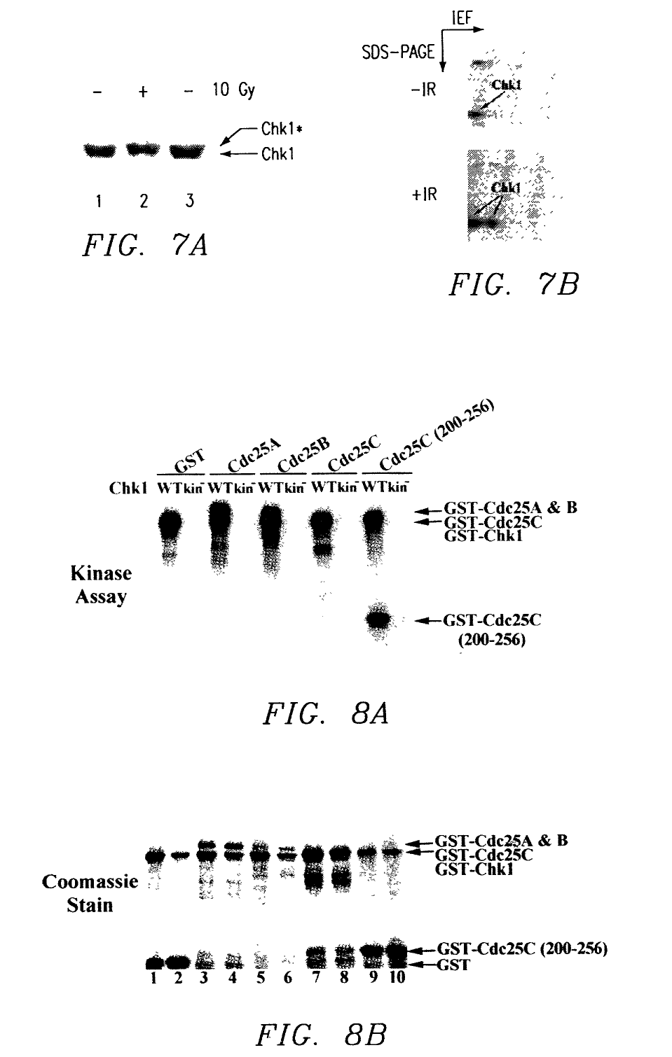

To determine whether hChkl is modified in response to DNA damage like spChkl, hChkl protein in extracts from cells treated with ionizing radiation was examined. hChk from extracts obtained from damaged cells showed a minor but reproducible reduction in mobility compared to hChkl from untreated cells (See, FIG. 7). The mobility alteration observed in response to DNA damage for spChkl was also slight, as previously reported (See e.g., N. C. Walworth et al., Nature 363:368 [19931; Al-Khodairy et al., Mol. Biol. Cell 5:147 [1994]; and N. C. Walworth and R. Bernards, Science 271:353 [1996]). This modification was confirmed by 2-dimensional gel analysis, which clearly demonstrated the generation of a more negatively charged hChkl species 2 hours after y-irradiation (See, FIG. 7). These results indicate that like spChkl, hChkl may participate in transduction of the DNA damage signal. Indirect immunofluorescence revealed that hChkl is localized to the nucleus in a punctate staining pattern, similar to that observed for ATM (See, G. Chen and E. Y. H. P. Lee, J. Biol. Chem., 271:33693 [1996]; and N. D. Lakin et al., Oncogene 13:2702 [1996]). mChkl expressed in BHK cells confirmed the nuclear localization.

To test for the ability of hChkl to regulate the cell cycle, hChkl or hChkl (D130A) (a catalytically inactive mutant), were transfected under the control of the CMV promoter, or the CMV vector alone into HeLa cells treated with and without 6 Gy of ionizing radiation. No perturbation of the cell cycle by either kinase relative to vector alone was observed, suggesting that overproduction alone was insufficient to deregulate the system.

Tyrosine phosphorylation of Cdc2 has been implicated in cell cycle arrest in response to DNA damage and replication blocks in both S. pombe (T. Enoch and P. Nurse, Cell 60:665 [1990]) and humans (P. Jin et aL., J. Cell Biol., 134:963 [1996]). In S. pombe, Cdc2 mutants that cannot be phosphorylated on tyrosine display an inability to arrest the cell cycle in response to blockade of DNA replication. Although it was originally thought that the DNA damage checkpoint did not operate through tyrosine phosphorylation, recent experiments have shown that tyrosine phosphorylation is required for S. pombe cells to arrest in response to DNA damage (P. Jin et al., J. Cell Biol., 134:963 [1996]). While it is now clear that tyrosine phosphorylation is required for proper checkpoint control, the experiments implicating tyrosine phosphorylation in this pathway do not distinguish between a regulatory role in which tyrosine phosphorylation rates are manipulated by the checkpoint pathways, or a passive role in which tyrosine phosphorylation is required to allow cell cycle arrest, but is not the actual target of the checkpoint pathway (S. Elledge, Science 274:1664 [1996]; and D. J. Lew and S. Kombluth, Curr. Opin. Cell. Biol., 8:795 [1996]).

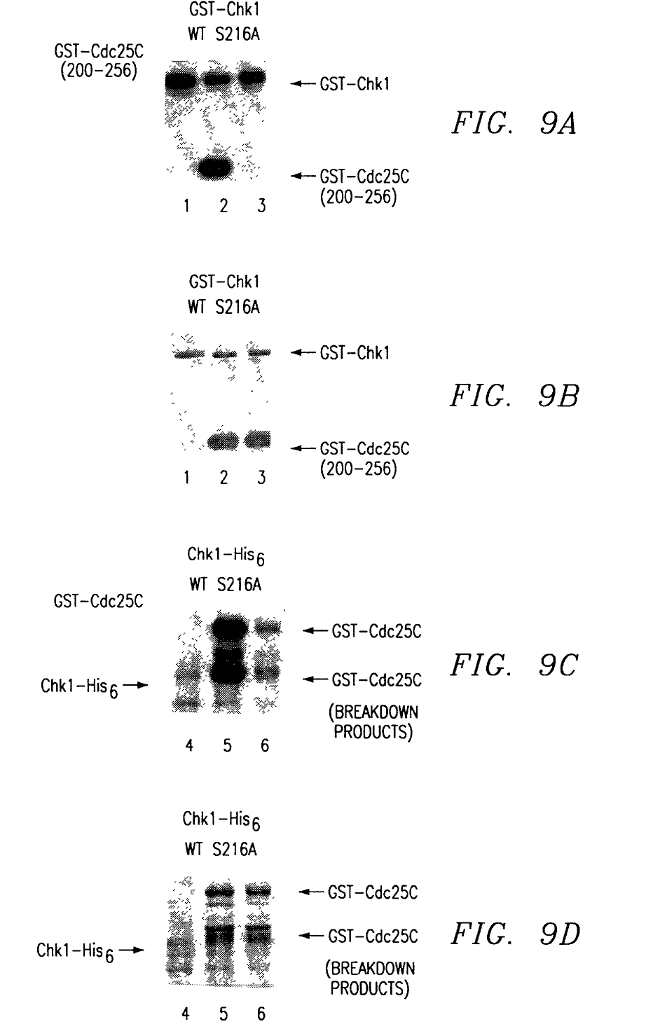

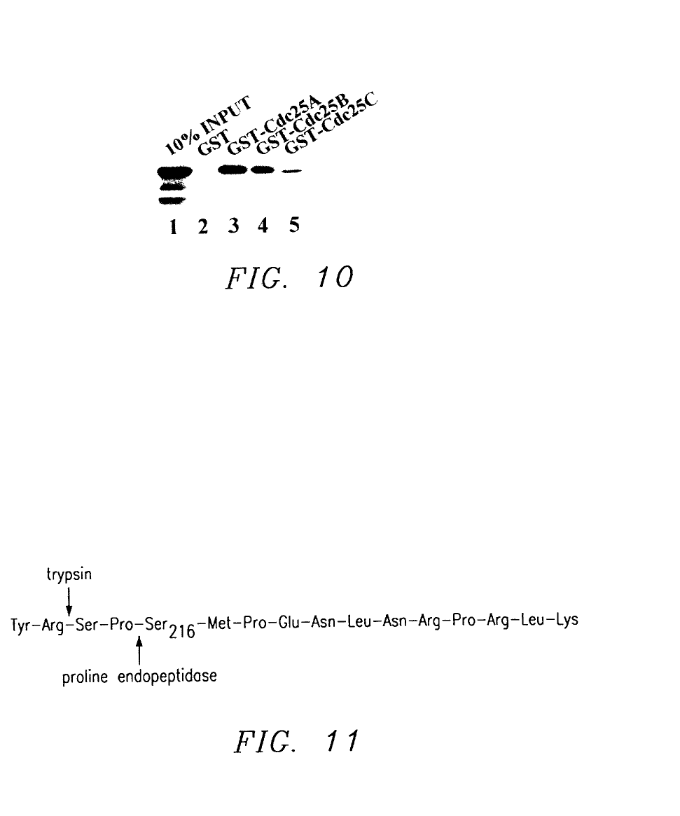

To address this issue, the ability of hCkl to phosphorylate key regulators of Cdk tyrosine phosphorylation was examined. For these experiments, the Cdc25 dualspecificity phosphatases, hCdc25A, hCdc25B, and hCdc25C were analyzed. These regulators were chosen for several reasons. First, overproduction of hCdk4 mutants in which the inhibitory tyrosine is changed to phenylalanine abrogates GI arrest in response to UV light (Y. Terada et al., Nature 376:358 [1995]). Secondly, the UV-sensitivity of chkl mutants in S. pombe is suppressed by inactivating cdc25 with a Ts (i.e., temperature sensitive) mutation (N. C. Walworth et al., Nature 363:368 [1993]). Finally, in S. pombe weelmik] mutants, DNA damage still causes a partial cell cycle delay that could be due to regulation of spCdc25 activity. GST-hChkl and GST-hChkl (D130A) were introduced into baculovirus, purified from baculovirus-infected insect cells, and incubated with GST-hCdc25A, GST-hCdc25B, and GST-hCdc25C, as described in Example 5. GST-hChkl phosphorylated all three Cdc25 proteins, but GST alone did not (See, FIG. 8). Although GST-hCdc25C co-migrated with GST-hChkl (which autophosphorylates), increased phosphorylation was observed at that position relative to phosphorylation in the presence of kinase alone, and phosphorylation of a GST-hCdc25C breakdown product was visible. In separate experiments using a His,-tagged hChkl derivative, there was clear phosphorylation of GST-hCdc25C (FIG. 9). A catalytically inactive mutant failed to phosphorylate itself or any of the Cdc25 proteins (See, FIG. 8).

Protein kinases often form complexes with their substrates. To examine determine whether this occurs with for hChkl and the Cdc25 proteins, GST-hCdc25 proteins on glutathione beads were incubated together with baculovirus extracts expressing His₆-tagged hChkl, and precipitated. GST-hCdc25A, GST-hCdc25B and GST-hCdc25C, each specifically bound hChkl, while GST alone did not (See, FIG. 10). Furthermore, two other GST fusion proteins, GST-Dunl and GST-Skpl, all failed to bind hChkl. These results indicate that Cdc25 can form complexes with hchk.

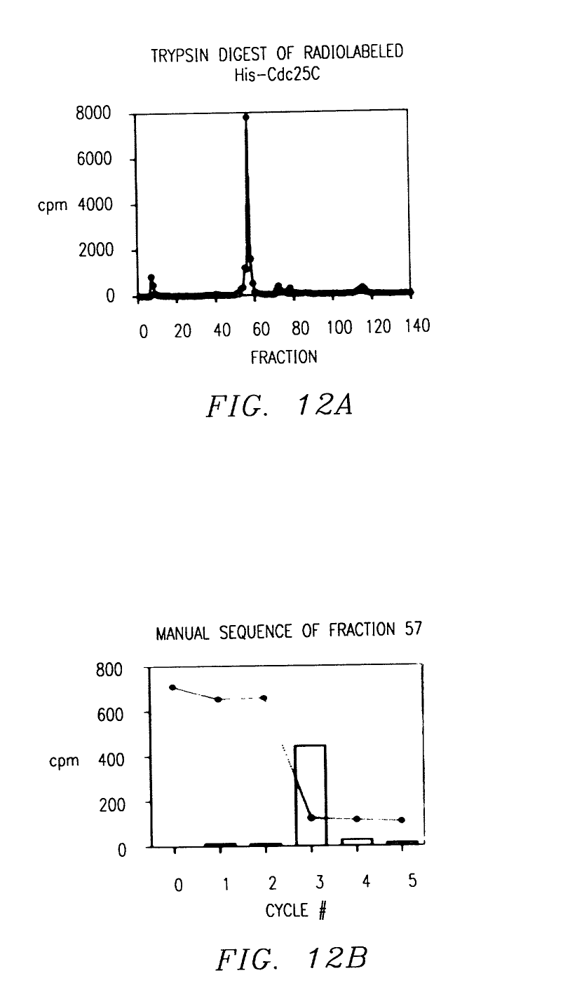

To establish the significance of the Cdc25 phosphorylation, the site of hChkl phosphorylation on Cdc25C was mapped. It was determined that Ser ²¹⁶ is the main site of phosphorylation of hCdc25C in vivo. hChkl phosphorylated a 56 amino acid region of the hCdc25C protein fused to GST, but not GST alone (FIGS. 8 and 9). This 56 amino acid motif contains four possible sites of phosphorylation. Peptide analysis of proteolytic fragments of full length HiS6-hCdc25C phosphorylated with GST-hChkl revealed a single phosphorylated tryptic peptide by high-pressure liquid chromatography. Edman degradation of this peptide indicated release of radioactivity in the third cycle (FIG. 12B). Further degradation of this tryptic fragment with proline endopeptidase resulted in production of a peptide that released radioactivity in the first cycle. The Serine²¹⁶ residue is the only site on hCdc25C that is consistent with this phosphorylation pattern (FIG. 11). To confirm this, the Cdc25C S216A mutation in GST-Cdc25C and Cdc25C(200-256) were constructed. It was determined that both were poor substrates for hChkl, confirming that Serine 216 is the site phosphorylation (FIG. 9). Serine²¹⁶ is also phosphorylated by spChkl, demonstrating phylogenetic conservation of this regulatory relationship.

The present invention provides evidence that the Chkl kinase family is conserved throughout eukaryotic evolution and that hChkl, like its S. pombe counterpart, is modified in response to DNA damage. This, together with the fact that ATM-related kinases are conserved members of checkpoint pathways and act upstream of chkl in S. pombe, suggests that this entire checkpoint pathway may be conserved in all eukaryotes. Nonetheless, the present invention provides the first mammalian Chkl sequences.

hcChkl directly phosphorylates a regulator of Cdc2 tyrosine phosphorylation, hCdc25C, on a physiologically significant residue, Serine2ll. In addition, overexpression of the hCdc25C S21 6A protein reduces the ability of cells to arrest in G2 in response to DNA damage, as observed previously for the Cdc2AF mutants. However, the overexpression studies alone do not prove that the DNA damage checkpoint pathway operates through tyrosine phosphorylation, because hyperactive Cdc2 may be able to bypass checkpoint control. Nonetheless, in combination with the fact that this inhibitory serine is directly phosphorylated by the DNA damageresponsive checkpoint kinase hCchkl, these results strongly imply that DNA damage regulates the G2-to-mitosis transition through control of Cdc2 tyrosine phosphorylation. These results suggest a model whereby in response to DNA damage, hChkl phosphorylates hCdc25C on Serine, leading to binding of 14-3-3 protein and inhibition of Cdc25C's ability to dephosphorylate and activate Cdc2. This model does not preclude a role for other cell cycle regulators such as Weel in the damage response. Although an understanding of the mechanism is not necessary in order to use the present invention, the facts that hChkl phosphorylated hCdc25A and hCdc25B, and that Serine²¹⁶ is conserved among these Cdc25 proteins, suggests that hChkl may regulate other DNA damage checkpoints, such as those controlling the GI to S phase transition, through a similar mechanism.

Definitions

To facilitate understanding of the invention, a number of terms are defined below.

Nucleic acid sequence” as used herein refers to an oligonucleotide, nucleotide, or polynucleotide, and fragments or portions thereof, and to DNA or RNA of genomic or synthetic origin which may be single- or double-stranded, and represent the sense or antisense strand. Similarly, “amino acid sequence” as used herein refers to an oligopeptide, peptide, polypeptide, or protein sequence, and fragments or portions thereof, and to naturally occurring or synthetic molecules.

A “composition comprising a given polynucleotide sequence” as used herein refers broadly to any composition containing the given polynucleotide sequence. The composition may comprise an aqueous solution. Compositions comprising polynucleotide sequences encoding human or murine Chkl (SEQ ID NOS: 3 and 4) or fragments thereof, may be employed as hybridization probes. In this case, the human and murine chkl-encoding polynucleotide sequences are typically employed in an aqueous solution containing salts (e.g., NACI), detergents (e.g., SDS) and other components (e.g., Denhardt's solution, dry milk, salmon sperm DNA, etc.).

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

“Peptide nucleic acid,” as used herein, refers to a molecule which comprises an oligomer to which an amino acid residue, such as lysine, and an amino group have been added. These small molecules, also designated anti-gene agents, stop transcript elongation by binding to their complementary strand of nucleic acid (Nielsen, P. E. et al., Anticancer Drug Des. 8:53-63 [1993]).

Chkl as used herein, refers to the amino acid sequences of substantially purified Chkl obtained from any species, particularly mammalian, including bovine, ovine, porcine, murine, equine, and human, from any source whether natural, synthetic, semi-synthetic, or recombinant. In particularly preferred embodiments, Chkl is human or murine.

“Consensus,” as used herein, refers to a nucleic acid sequence which has been resequenced to resolve uncalled bases, or which has been extended using any suitable method known in the art, in the 5′ and/or the 3′ direction and resequenced, or which has been assembled from the overlapping sequences of more than one clone using any suitable method known in the art, or which has been both extended and assembled.

A “variant” of human or murine Chkl, as used herein, refers to an amino acid sequence that is altered by one or more amino acids. The variant may have conservative” changes, wherein a substituted amino acid has similar structural or chemical properties (e.g., replacement of leucine with isoleucine). More rarely, a variant may have “nonconservative” changes (e.g., replacement of a glycine with a tryptophan). Similar minor variations may also include amino acid deletions or insertions, or both. Guidance in determining which amino acid residues may be substituted, inserted, or deleted without abolishing biological or immunological activity may be found using computer programs well known in the art, for example, DNASTAR software.

A “deletion,” as used herein, refers to a change in either amino acid or nucleotide sequence in which one or more amino acid or nucleotide residues, respectively, are absent.

An “insertion” or “addition,” as used herein, refers to a change in an amino acid or nucleotide sequence resulting in the addition of one or more amino acid or nucleotide residues, respectively, as compared to the naturally occurring molecule.

A “substitution,” as used herein, refers to the replacement of one or more amino acids or nucleotides by different amino acids or nucleotides, respectively.

The term “biologically active,” as used herein, refers to a protein having structural, regulatory, or biochemical functions of a naturally occurring molecule. Likewise, “immunologically active” refers to the capability of the natural, recombinant, or synthetic human or murine Chkl, or any oligopeptide thereof, to induce a specific immune response in appropriate animals or cells and to bind with specific antibodies.

The term “modulate,” as used herein, refers to a change or an alteration in the biological activity of human or murine Chkl. Modulation may be an increase or a decrease in protein activity, a change in binding characteristics, or any other change in the biological, functional, or immunological properties of human or murine Chkl. The term “mimetic,” as used herein, refers to a molecule, the structure of which is developed from knowledge of the structure of Chkl, or portions thereof and, as such, is able to effect some or all of the actions of human or murine Chkl-like molecules.

The term “derivative,” as used herein, refers to the chemical modification of a nucleic acid encoding human or murine Chkl, or the encoded human or murine Chkl protein. Illustrative of such modifications would be replacement of hydrogen by an alkyl, acyl, or amino group. A nucleic acid derivative would encode a polypeptide which retains essential biological characteristics of the natural molecule.

The term “substantially purified,” as used herein, refers to nucleic or amino acid sequences that are removed from their natural environment, isolated or separated, and are at least 60% free, preferably 75% free, and most preferably 90% free from other components with which they are naturally associated.

The term “hybridization,” as used herein, refers to any process by which a strand of nucleic acid binds with a complementary strand through base pairing. Hybridization and the strength of hybridization (i.e., the strength of the association between the nucleic acids) is impacted by such factors as the degree of complementary between the nucleic acids, stringency of the conditions involved, the T of the formed hybrid, and the G:C ratio within the nucleic acids.

As used herein, the term “T_{m”} is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(%G+C), when a nucleic acid is in aqueous solution at 1 M NaCl (see e.g., Anderson and Young, Quantitative Filter Hybridization, in Nucleic Acid Hybridization (1985). Other references include more sophisticated computations which take structural as well as sequence characteristics into account for the calculation of Tₘ.

The term “hybridization complex,” as used herein, refers to a complex formed between two nucleic acid sequences by virtue of the formation of hydrogen binds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C₀t or R₀t analysis) or between one nucleic acid sequence present in solution and another nucleic acid sequence immobilized on a solid support (e.g., membranes, filters, chips, pins or glass slides to which cells have been fixed for in situ hybridization).

The terms “complementary” or “complementarity,” as used herein, refer to the natural binding of polynucleotides under permissive salt and temperature conditions by base-pairing. For example, for the sequence “A-G-T” binds to the complementary sequence “T-C-A”. Complementarity between two single-stranded molecules may be “partial”, in which only some of the nucleic acids bind, or it may be complete when total complementarity exists between the single stranded molecules. The degree of complementarity between nucleic acid strands has significant effects on the efficiency and strength of hybridization between nucleic acid strands. This is of particular importance in amplification reactions, which depend upon binding between nucleic acids strands.

The term “homology,” as used herein, refers to a degree of complementarity. There may be partial homology or complete homology (i.e., identity). A partially complementary sequence is one that at least partially inhibits an identical sequence from hybridizing to a target nucleic acid; it is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence or probe to the target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding, the probe will not hybridize to the second non-complementary target sequence. When used in reference to a single-stranded nucleic acid sequence, the ten-n “substantially homologous” refers to any probe which can hybridize (i.e., it is the complement of) the single-stranded nucleic acid sequence under conditions of low stringency as described.

As known in the art, numerous equivalent conditions may be employed to comprise either low or high stringency conditions. Factors such as the length and nature (DNA, RNA, base composition) of the sequence, nature of the target (DNA, RNA, base composition, presence in solution or immobilization, etc.), and the concentration of the salts and other components (e.g., the presence or absence of formamide, dextran sulfate and/or polyethylene glycol) are considered and the hybridization solution may be varied to generate conditions of either low or high stringency different from, but equivalent to, the above listed conditions. As used herein the term “stringency” is used in reference to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences. Thus, conditions of “weak” or “low” stringency are often required with nucleic acids that are derived from organisms that are genetically diverse, as the frequency of complementary sequences is usually less.

Low stringency conditions comprise conditions equivalent to binding or hybridization at 42° C. in a solution consisting of 5×SSPE (43.8 g/l NaCl, 6.9 g/l NaH₂PO₄?H₂0 and 1.85 g/l EDTA, pH adjusted to 7.4 with NaOH), 0.1% SDS, 5×Denhardt's reagent (50×Denhardt's contains per 500 ml: 5 g Ficoll (Type 400, Pharamcia), 5 g BSA [Fraction V; Sigma]) and 100 i g/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions which promote hybridization under conditions of high stringency (e.g., increasing the temperature of the hybridization and/or wash steps, the use of formamide in the hybridization solution, etc.).

The term “antisense,” as used herein, refers to nucleotide sequences which are complementary to a specific DNA or RNA sequence. The term “antisense strand” is used in reference to a nucleic acid strand that is complementary to the “sense” strand. Antisense molecules may be produced by any method, including synthesis by ligating the gene(s) of interest in a reverse orientation to a viral promoter which permits the synthesis of a complementary strand. Once introduced into a cell, this transcribed strand combines with natural sequences produced by the cell to form duplexes. These duplexes then block either the further transcription or translation. In this manner, mutant phenotypes may be generated. The designation “negative” is sometimes used in reference to the antisense strand, and “positive” is sometimes used in reference to the sense strand.

The term also is used in reference to RNA sequences which are complementary to a specific RNA sequence (e.g., MRNA). Included within this definition are antisense RNA (“asRNA”) molecules involved in gene regulation by bacteria. Antisense RNA may be produced by any method, including synthesis by splicing the gene(s) of interest in a reverse orientation to a viral promoter which permits the synthesis of a coding strand. Once introduced into an embryo, this transcribed strand combines with natural MRNA produced by the embryo to form duplexes. These duplexes then block either the further transcription of the MRNA or its translation. In this manner, mutant phenotypes may be generated. The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

A gene may produce multiple RNA species which are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

The term “portion,” as used herein, with regard to a protein (as in “a portion of a given protein”) refers to fragments of that protein. The fragments may range in size from four amino acid residues to the entire amino acid sequence minus one amino acid. Thus, a protein “comprising at least a portion of the amino acid sequence of SEQ ID NO: 3″ encompasses the full-length human Chkl, and fragments thereof.

Transformation,” as defined herein, describes a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome.

The term “transfection” as used herein refers to the introduction of foreign DNA into eukaryotic cells. Transfection may be accomplished by a variety of means known to the art including calcium phosphate-DNA co-precipitation, DEAE-dextranmediated transfection, polybrene-mediated transfection, electroporation, microinjection, liposome fusion, lipofection, protoplast fusion, retroviral infection, and biolistics. Thus, the term “stable transfection” or “stably transfected” refers to the introduction and integration of foreign DNA into the genome of the transfected cell. The term “stable transfectant” refers to a cell which has stably integrated foreign DNA into the genomic DNA. The term also encompasses cells which transiently express the inserted DNA or RNA for limited periods of time. Thus, the term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells which have taken up foreign DNA but have failed to integrate this DNA.

The term “antigenic determinant,” as used herein, refers to that portion of a molecule (i.e., an antigen) that makes contact with a particular antibody (i.e., an epitope). When a protein or fragment of a protein is used to immunize a host animal (e.g., an “immunocompent” animal with “immunocompetent cells”), numerous regions of the protein may induce the production of antibodies which bind specifically to a given region or three-dimensional structure on the protein; these regions or structures are referred to as antigenic determinants. An antigenic determinant may compete with the intact antigen (i.e., the immunogen used to elicit the immune response) for binding to an antibody.

The terms “specific binding” or “specifically binding,” as used herein, in reference to the interaction of an antibody and a protein or peptide, mean that the interaction is dependent upon the presence of a particular structure (i.e., the antigenic determinant or epitope) on the protein; in other words, the antibody is recognizing and binding to a specific protein structure rather than to proteins in general. For example, if an antibody is specific for epitope “A”, the presence of a protein containing epitope A (or free, unlabeled A) in a reaction containing labeled “A” and the antibody will reduce the amount of labeled A bound to the antibody.

The term “sample,” as used herein, is used in its broadest sense. The term encompasses biological sample(s) suspected of containing nucleic acid encoding human or murine Chkl or fragments thereof, and may comprise a cell, chromosomes isolated from a cell (e.g., a spread of metaphase chromosomes), genomic DNA (in solution or bound to a solid support such as for Southern analysis), RNA (in solution or bound to a solid support such as for northern analysis), CDNA (in solution or bound to a solid support), an extract from cells or a tissue, and the like.

The term “correlates with expression of a polynucleotide,” as used herein, indicates that the detection of the presence of ribonucleic acid that is similar to SEQ ID NO: 1 or 2, by Northern analysis is indicative of the presence of mRNA encoding human or murine Chkl, in a sample and thereby correlates with expression of the transcript from the polynucleotide encoding the protein.

“Alterations” in the polynucleotide of SEQ ID NO: 1 or 2, as used herein, comprise any alteration in the sequence of polynucleotides encoding human or murine Chkl, respectively, including deletions, insertions, and point mutations that may be detected using hybridization assays. Included within this definition is the detection of alterations to the genomic DNA sequence which encodes human Chkl (e.g., by alterations in the pattern of restriction fragment length polymorphisms capable of hybridizing to SEQ ID NO: 1), the inability of a selected fragment of SEQ ID NO: 1 to hybridize to a sample of genomic DNA (e.g., using allele-specific oligonucleotide probes), and improper or unexpected hybridization, such as hybridization to a locus other than the normal chromosomal locus for the polynucleotide sequence encoding human Chkl (e.g., using fluorescent in situ hybridization [FISH] to metaphase chromosomes spreads).

As used herein, the term “antibody” (or “immunoglobulin”), refers to intact molecules as well as fragments thereof, such as Fa, F(ab′)₂, and Fv, which are capable of binding the epitopic determinant. Antibodies that bind human or murine Chkl polypeptides can be prepared using intact polypeptides or fragments containing small peptides of interest as the immunizing antigen. The polypeptide or peptide used to immunize an animal can be derived from the transition of RNA or synthesized chemically, and can be conjugated to a carrier protein, if desired. Commonly used carriers that are chemically coupled to peptides include bovine serum albumin and thyroglobulin. The coupled peptide is then used to immunize the animal (e.g., a mouse, a rat, or a rabbit).

The term “humanized antibody,” as used herein, refers to antibody molecules in which amino acids have been replaced in the non-antigen binding regions in order to more closely resemble a human antibody, while still retaining the original binding ability.

As used herein, the term “polyA⁺ RNA” refers to RNA molecules having a stretch of adenine nucleotides at the 3′ end. This polyadenine stretch is also referred to as a “poly-A tail”. Eukaryotic MRNA molecules contain poly-A tails and are referred to as polyA⁺ RNA.

As used herein, the term “fusion protein” refers to a chimeric protein containing the protein of interest (i.e., mouse or human chkl and fragments thereof) joined to an exogenous protein fragment (the fusion partner which consists of a non-chkl protein). The fusion partner may enhance solubility of the chkl protein as expressed in a host cell, may provide an affinity tag to allow purification of the recombinant fusion protein from the host cell or culture supernatant, or both. If desired, the fusion protein may be removed from the protein of interest (i.e., chkl protein or fragments thereof) by a variety of enzymatic or chemical means known to the art.

As used herein, the term “affinity tag” refers to such structures as a “polyhistidine tract” or “poly-histidine tag,” or any other structure or compound which facilitates the purification of a recombinant fusion protein from a host cell, host cell culture supernatant, or both. As used herein, the term “flag tag” refers to short polypeptide marker sequence useful for recombinant protein identification and purification.

As used herein, the terms “poly-histidine tract” and “poly-histidine tag,” when used in reference to a fusion protein refers to the presence of two to ten histidine residues at either the amino- or carboxy-terminus of a protein of interest. A poly-histidine tract of six to ten residues is preferred. The poly-histidine tract is also defined functionally as being a number of consecutive histidine residues added to the protein of interest which allows the affinity purification of the resulting fusion protein on a nickel-chelate or IDA column.

As used herein, the term “chimeric protein” refers to two or more coding sequences obtained from different genes, that have been cloned together and that, after translation, act as a single polypeptide sequence. Chimeric proteins are also referred to as “hybrid proteins.” As used herein, the term “chimeric protein” refers to coding sequences that are obtained from different species of organisms, as well as coding sequences that are obtained from the same species of organisms.

As used herein, the term “protein of interest” refers to the protein whose expression is desired within the fusion protein. In a fusion protein, the protein of interest will be joined or fused with another protein or protein domain, the fusion partner, to allow for enhanced stability of the protein of interest and/or ease of purification of the fusion protein.

As used herein, the term “abnormal Chkl protein” refers to Chkl that lacks function (i.e., does not function as Chkl protein in normal cells), or is not recognized by sequences encoding full-length functional Chkl protein.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro.

As used herein, the term “selectable marker” refers to the use of a gene which encodes an enzymatic activity that confers the ability to grow in medium lacking what would otherwise be an essential nutrient (e.g. the HIS3 gene in yeast cells); in addition, a selectable marker may confer resistance to an antibiotic or drug upon the cell in which the selectable marker is expressed. Selectable markers may be “dominant”; a dominant selectable marker encodes an enzymatic activity which can be detected in any eukaryotic cell line. Examples of dominant selectable markers include the bacterial aminoglycoside 3′ phosphotransferase gene (also referred to as the neo gene) which confers resistance to the drug G418 in mammalian cells, the bacterial hygromycin G phosphotransferase (hyg) gene which confers resistance to the antibiotic hygromycin and the bacterial xanthine-guanine phosphoribosyl transferase gene (also referred to as the gpt gene) which confers the ability to grow in the presence of mycophenolic acid. Other selectable markers are not dominant in that there use must be in conjunction with a cell line that lacks the relevant enzyme activity. Examples of non-dominant selectable markers include the thymidine kinase (tk) gene which is used in conjunction with tk cell lines, the CAD gene which is used in conjunction with CAD-deficient cells and the mammalian hypoxanthine-guanine phosphoribosyl transferase (hprt) gene which is used in conjunction with hprr cell lines. A review of the use of selectable markers in mammalian cell lines is provided in Sambrook, J. et aL, Molecular Cloning: A Laboratory Manual, 2nd ed., Cold Spring Harbor Laboratory Press, New York (1989) pp.16.9-16.15.

As used herein, the term “vector” is used in reference to nucleic acid molecules that transfer DNA segment(s) from one cell to another. The term “vehicle” is sometimes used interchangeably with “vector.”

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

As used herein, the term “arnplifiable nucleic acid” is used in reference to nucleic acids which may be amplified by any amplification method. It is contemplated that “arnplifiable nucleic acid” will usually comprise “sample template.”

As used herein, the term “sample template” refers to nucleic acid originating from a sample which is analyzed for the presence of “target” (defined below). In contrast, “background template” is used in reference to nucleic acid other than sample template which may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labelled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target,” when used in reference to the polymerase chain reaction, refers to the region of nucleic acid bounded by the primers used for polymerase chain reaction. Thus, the “target” is sought to be sorted out from other nucleic acid sequences. A “segment” is defined as a region of nucleic acid within the target sequence.

As used herein, the term “polymerase chain reaction” (“PCR”) refers to the method of K. B. Mullis U.S. Pat. Nos. 4,683,195 4,683,202, and 4,965,188, hereby incorporated by reference, which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”.

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of “P-labeled deoxynucleotide triphosphates, such as DCTP or DATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications.

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Qβ replicase, MDV-1 RNA is the specific template for the replicase (D. L. Kacian et al., Proc. Natl. Acad. Sci. USA 69:3038 [1972]). Other nucleic acid will not be replicated by this amplification enzyme. Similarly, in the case of T7 RNA polymerase, this amplification enzyme has a stringent specificity for its own promoters (M. Chamberlin et al., Nature 228:227 [1970]). In the case of T4 DNA ligase, the enzyme will not ligate the two oligonucleotides where there is a mismatch between the oligonucleotide substrate and the template at the ligation junction (D. Y. Wu and R. B. Wallace, Genomics 4:560 [1989]). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press [1989]).

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “amplification reagents” refers to those reagents (deoxyribonucleotide triphosphates, buffer, etc.), needed for amplification except for primers, nucleic acid template and the amplification enzyme. Typically, amplification reagents along with other reaction components are placed and contained in a reaction vessel (test tube, microwell, etc.).

As used herein, the terms “restriction endonucleases” and “restriction enzymes” refer to bacterial enzymes, each of which cut double-stranded DNA at or near a specific nucleotide sequence.

As used herein, the term “recombinant DNA molecule” as used herein refers to a DNA molecule which is comprised of segments of DNA joined together by means of molecular biological techniques.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotides referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region However, enhancer elements can exert their effect even when located 3′ of the promoter element and the coding region. Transcription termination and polyadenylation signals are located 3′ or downstream of the coding region.

As used herein, the term “an oligonucleotide having a nucleotide sequence encoding a gene” means a nucleic acid sequence comprising the coding region of a gene or in other words the nucleic acid sequence which encodes a gene product. The coding region may be present in either a CDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc. (defined infta).

Transcriptional control signals in eukaryotes comprise “promoter” and “enhancer” elements. Promoters and enhancers consist of short arrays of DNA sequences that interact specifically with cellular proteins involved in transcription (T. Maniatis et al., Science 236:1237 [1987]). Promoter and enhancer elements have been isolated from a variety of eukaryotic sources including genes in yeast, insect and mammalian cells and viruses (analogous control elements, i.e., promoters, are also found in prokaryote). The selection of a particular promoter and enhancer depends on what cell type is to be used to express the protein of interest. Some eukaryotic promoters and enhancers have a broad host range while others are functional in a limited subset of cell types (for review see, S. D. Voss et al.,Trends Biochem. Sci., 11:287 [1986]; and T. Maniatis et al., supra). For example, the SV40 early gene enhancer is very active in a wide variety of cell types from many mammalian species and has been widely used for the expression of proteins in mammalian cells (R. Dijkema et al., EMBO J. 4:761 [19851). Two other examples of promoter/enhancer elements active in a broad range of manu-nalian cell types are those from the human elongation factor lot gene (T. Uetsuki et al., J. Biol. Chem., 264:5791 [1989]; D. W. Kim et al., Gene 91:217 [1990]; and S. Mizushima and S. Nagata, Nuc. Acids. Res., 18:5322 [19901) and the long terminal repeats of the Rous sarcoma virus (C. M. Gorman et al., Proc. Natl. Acad. Sci. USA 79:6777 [1982]) and the human cytomegalovirus (M. Boshart et aL, Cell 41:521 [1985]).

As used herein, the term “promoter/enhancer” denotes a segment of DNA which contains sequences capable of providing both promoter and enhancer functions (i.e., the functions provided by a promoter element and an enhancer element, see above for a discussion of these functions). For example, the long terminal repeats of retroviruses contain both promoter and enhancer functions. The enhancer/promoter may be endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

The presence of “splicing signals” on an expression vector often results in higher levels of expression of the recombinant transcript. Splicing signals mediate the removal of introns from the primary RNA transcript and consist of a splice donor and acceptor site (J. Sambrook et aL, Molecular Cloning: A Laboratory Manual, 2nd ed., Cold Spring Harbor Laboratory Press, New York [1989], pp. 16.7-16.8). A commonly used splice donor and acceptor site is the splice junction from the 16S RNA of SV40.

Efficient expression of recombinant DNA sequences in eukaryotic cells requires expression of signals directing the efficient termination and polyadenylation of the resulting transcript. Transcription termination signals are generally found downstream of the polyadenylation signal and are a few hundred nucleotides in length. The term “poly A site” or “poly A sequence” as used herein denotes a DNA sequence which directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be “heterologous” or “endogenous.” An endogenous poly A signal is one that is found naturally at the 3′ end of the coding region of a given gene in the genome. A heterologous poly A signal is one which is one which is isolated from one gene and placed 3′ of another gene. A commonly used heterologous poly A signal is the SV40 poly A signal. The SV40 poly A signal is contained on a 237 bp BamHllBcll restriction fragment and directs both termination and polyadenylation (J. Sambrook, supra, at 16.6-16.7).

Eucaryotic expression vectors may also contain “viral replicons” or “viral origins of replication.” Viral replicons are viral DNA sequences which allow for the extrachromosomal replication of a vector in a host cell expressing the appropriate replication factors. Vectors which contain either the SV40 or polyoma virus origin of replication replicate to high copy number (up to 104 copies/cell) in cells that express the appropriate viral T antigen. Vectors which contain the replicons from bovine papillomavirus or Epstein-Barr virus replicate extrachromosomally at low copy number (−100 copies/cell).

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

The term “calcium phosphate co-precipitation” refers to a technique for the introduction of nucleic acids into a cell. The uptake of nucteic acids by cells is enhanced when the nucleic acid is presented as a calcium phosphate-nucleic acid coprecipitate. The original technique of Graham and van der Eb (Graham and van der Eb, Virol., 52:456 [1973]), has been modified by several groups to optimize conditions for particular types of cells. The art is well aware of these numerous modifications.

The term “Southern blot” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al. Molecular Cloning: A Laboratory Manual, Cold Spring Harbor Press, NY, pp 9.31-9.58 [1989]).

The term “Northern blot” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, J. et al., supra, pp 7.39-7.52 [1989]).

The term “YIp plasmid” refers to yeast integrating plasmids which contain selectable yeast genes but lack sequences which allow for the autonomous replication of the plasmid in a yeast cell. Transformation of the host yeast cell occurs by integration of the YIp plasmid into the yeast genome. This integration occurs by recombination between yeast sequences present on the YIp plasmid and homologous sequences present in the genome.

The term “prototrophic” or “prototrophy” refers to an organism that can survive growth under conditions where one or more essential nutrients are lacking in the growth medium. For example, if a yeast cell is transformed to histidine prototrophy, this means that the yeast cell now contains gene sequences encoding the enzyme necessary for the production of the amino acid histidine; therefore, the transformed yeast cell does not require the presence of histidine in the growth medium.

The term “isolated” when used in relation to a nucleic acid, as in “an isolated oligonucleotide” refers to a nucleic acid sequence that is identified and separated from at least one containinant nucleic acid with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific MRNA sequence encoding a specific protein, are found in the cell as a mixture with numerous other MRNA s which encode a multitude of proteins. However, isolated nucleic acid encoding a mammalian Chkl protein includes, by way of example, such nucleic acid in cells ordinarily expressing a Chkl protein where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may single-stranded), but may contain both the sense and anti-sense strands (i.e., the oligonucleotide may be double-stranded).

As used herein, a “portion of a chromosome” refers to a discrete section of the chromosome. Chromosomes are divided into sites or sections by cytogenetists as follows: the short (relative to the centromere) arm of a chromosome is termed the “p” arm; the long arm is termed the “q” arm. Each arm is then divided into 2 regions termed region 1 and region 2 (region 1 is closest to the centromere). Each region is further divided into bands. The bands may be further divided into sub-bands. For example, the 11p15.5 portion of human chromosome 11 is the portion located on chromosome 11 (11) on the short arm (p) in the first region (1) in the 5th band (5) in sub-band 5 (0.5). A portion of a chromosome may be “altered;” for instance the entire portion may be absent due to a deletion or may be rearranged (e.g., inversions, translocations, expanded or contracted due to changes in repeat regions). In the case of a deletion, an attempt to hybridize (i.e., specifically bind) a probe homologous to a particular portion of a chromosome could result in a negative result (i.e., the probe could not bind to the sample containing genetic material suspected of containing the missing portion of the chromosome). Thus, hybridization of a probe homologous to a particular portion of a chromosome may be used to detect alterations in a portion of a chromosome.

The term “sequences associated with a chromosome” means preparations of chromosomes (e.g., spreads of metaphase chromosomes), nucleic acid extracted from a sample containing chromosomal DNA (e.g., preparations of genomic DNA); the RNA which is produced by transcription of genes located on a chromosome (e.g., hnRNA and mRNA) and cDNA copies of the RNA transcribed from the DNA located on a chromosome. Sequences associated with a chromosome may be detected by numerous techniques including probing of Southern and Northern blots and in situ hybridization to RNA, DNA, or metaphase chromosomes with probes containing sequences homologous to the nucleic acids in the above listed preparations.

As used herein the term “coding region” when used in reference to structural gene refers to the nucleotide sequences which encode the amino acids found in the nascent polypeptide as a result of translation of a mRNA molecule. The coding region is bounded, in eukaryotes, on the 5′ side by the nucleotide triplet “ATG” which encodes the initiator methionine and on the 3′ side by one of the three triplets which specify stop codons (i.e., TAA, TAG, TGA).

As used herein, the term “structural gene” refers to a DNA sequence coding for RNA or a protein. In contrast, “regulatory genes” are structural genes which encode products which control the expression of other genes (e.g., transcription factors).

As used herein, the term “gene” means the deoxyribonucleotide sequences comprising the coding region of a structural gene and the including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences; these sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, post-transcriptional cleavage and polyadenylation.

As used herein, the term “purified” or “to purify” refers to the removal of contaminants from a sample. For example, anti-chkl antibodies are purified by removal of contaminating non-immunoglobulin proteins; they are also purified by the removal of immunoglobulin that does not bind chkl. The removal of nonimmunoglobulin proteins and/or the removal of immunoglobulins that do not bind chkl results in an increase in the percent of chkl-reactive immunoglobulins in the sample. In another example, recombinant chkl polypeptides are expressed in bacterial host cells and the polypeptides are purified by the removal of host cell proteins; the percent of recombinant chkl polypeptides is thereby increased in the sample.

The term “recombinant DNA molecule” as used herein refers to a DNA molecule which is comprised of segments of DNA joined together by means of molecular biological techniques.

The term “recombinant protein” or “recombinant polypeptide” as used herein refers to a protein molecule which is expressed from a recombinant DNA molecule.

The term “native protein” as used herein to indicate that a protein does not contain amino acid residues encoded by vector sequences; that is the native protein contains only those amino acids found in the protein as it occurs in nature. A native protein may be produced by recombinant means or may be isolated from a naturally occurring source.

As used herein, the term “chkl ” protein” or “chkl protein sequence” refers to a protein which is encoded by a chkl gene sequence or to a protein.

As used herein, the term “non-chkl protein” or “non-chkl protein sequence” refers to that portion of a fusion protein which comprises a protein or protein sequence which is not derived from a chkl protein.

### Experimental

The following examples are provided in order to demonstrate and further illustrate certain preferred embodiments and aspects of the present invention and are not to be construed as limiting the scope thereof.

In the experimental disclosure which follows, the following abbreviations apply: h (human); sc (Saccharomyces cerevisiae); sp (Schizosaccharomyces pombe); ce (Caenhorrhabditis elegans); dm (Drosophila melanogaster); ° C. (degrees Centigrade); rpm (revolutions per minute); BSA (bovine serum albumin); CFA (complete Freund's adjuvant); IFA (incomplete Freund's _{adjuvant}); IgG (immunoglobulin G); IM (intramuscular); IP (intraperitoneal); IV (intravenous or intravascular); SC (subcutaneous); H2O(water); HCl (hydrochloric acid); aa (arnino acid); bp (base pair); kb (kilobase pair); kD (kilodaltons); gm (grams); μg (micrograms); mg (milligrams); ng (nanograins); μl (microliters); ml (milliliters); mm (millimeters); mn (manometers); μm (micrometer); M (molar); mM (millimolar); MW (molecular weight); sec (seconds); min(s) (minute/minutes); hr(s) (hour/hours); MgCl₂ (magnesium chloride); NaCl (sodium chloride); OD₂₈₀ (optical density at 280 nm); OD₆₀₀ (optical density at 600 nm); PAGE (polyacrylamide gel electrophoresis); PBS (phosphate buffered saline [ 150 mM NaCl, 10 mM sodium phosphate buffer, pH 7.2]); PEG (polyethylene glycol); PMSF (phenylmethylsulfonyl fluoride); SDS (sodium dodecyl sulfate); Tris (tris(hydroxymethyl)aminomethane); w/v (weight to volume); v/v (volume to volume);

Amersham (Amersham Life Science, Inc. Arlington Heights, Ill.); ICN (ICN Pharmaceuticals, Inc., Costa Mesa, Calif.); Amicon (Amicon, Inc., Beverly, Mass.); ATCC (American Type Culture Collection, Rockville, Md.); Becton Dickinson (Becton Dickinson Labware, Lincoln Park, N.J.); BioRad (BioRad, Richmond, Calif.); Clontech (CLONTECH Laboratories, Palo Alto, Calif.); Difco (Difco Laboratories, Detroit, Mich.); GIBCO BRL or Gibco BRL (Life Technologies, Inc., Gaithersburg, Md.); Invitrogen (Invitrogen Corp., San Deigo, Calif.); Kodak (Eastman Kodak Co., New Haven, Conn.); New England Biolabs (New England Biolabs, Inc., Beverly, Mass.); Novagen (Novagen, Inc., Madison, Wis.); Pharmacia (Pharmacia, Inc., Piscataway, N.J.); Sigma (Sigma Chemical Co., St. Louis, Mo.); Sorvall (Sorvall Instruments, a subsidary of DuPont Co., Biotechnology Systems, Wilmington, Del.); Stratagene (Stratagene Cloning Systems, La Jolla, Calif.); Whatman (Whatman LabSales, Hillsboro, Oreg.); Bethyl Laboratories (Bethyl Laboratories, Montgomery, Tex.); and Zeiss (Carl Zeiss, Inc., Thornwood, N.Y.).

Unless otherwise indicated, all restriction enzymes were obtained from New England BioLabs and were used according to the manufacturer's instructions; all oligonucleotide primers, adapter and linkers were synthesized using standard methodologies on an ABI DNA synthesizer. All chemicals were obtained from Sigma unless otherwise indicated.

### EXAMPLE 1

### Human and Murine Chkl

In this Example, human and murine checkpoint genes were identified by searching for homologs of yeast checkpoint genes. In the experiments to identify the human homolog, degenerate PCR primers to conserved motifs in the kinase domains of spChkl were used to screen a human B cell library by PCR, as known in the art.

A DNA fragment containing the ORF for the Chkl gene was obtained using the polymerase chain reaction (PCR) as follows. The DNA was obtained from human peripheral blood lymphocytes. Gel-purified degenerate primers used in the reaction were 5′-GGNGGNGAGT/CT/CTNATGGAT/CTT-3′ (SEQ ID NO: 5) and 5 ′-TTGGACAGGCCAAAGTC-3′ (SEQ ID NO: 6). The reaction conditions comprised the following steps: denaturation at 95° C. for 5 minutes, 80° C. for 1 minute, during which Taq was added, and thirty cycles of 95° C. for 30 seconds; 52° C. for 30 seconds; and 72° C. for 2 minutes.

Four of 35 clones showed similarity to spChkl, and one clone was used to probe 2×10⁵ plaques from a λACT human B cell cDNA library. This probe was also used in subsequent experiments (it is referred to as the “PCR probe”). A partial sequence of this probe was determined and is shown below:

(5′gggggggagctgtttgaccgaatagagccagacataggcatgcctgaaccagatgctcagagattcttccatca
actcatgggaggggtggtttatctgcatggtattggaataactcacagggatattaaaccagaaaatcttctgttggaag
aaagggataacctcaaaatctcagactttggc-3′)
(SEQ ID NO: 11).

The library was constructed using methods known in the art (See, T. Durfee et al., Genes Develop., 7:555-569 [1993]), and deposited with the ATCC (ATCC Accession No. ATC 87003). The library was screened by hybridizing DNA present on filters with radiolabeled PCR probe (described above) in Hybridization Solution 1 (48% formamide, 5×SSC, 20 mM Tris-Cl, pH 7.6, 1×Denhardt's solution, 10% dextran sulfate, and 0.1% SDS) at 42° C. overnight. The filters were washed three times in low stringency wash (2×SSC/0.1% SDS) for 10 minutes at room temperature, and twice in high stringency wash (0.2×SSC/0 .I% SDS) at 65° C., and then exposed for autoradiography.

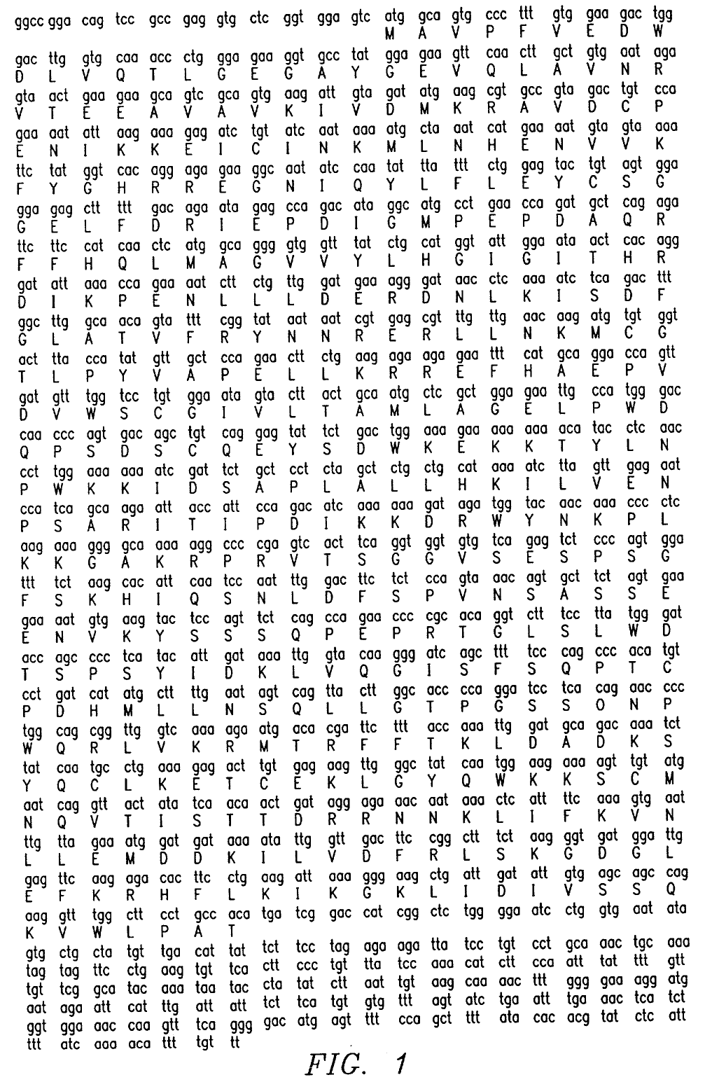

The plaques showing hybridization to probe were isolated and used to infect a bacterial strain expressing the Cre enzyme. The recombinant plasmids containing the cDNA inserts were purified and screened by Southern analyses using the PCR-generated probe described and used above. Probing of this library resulted in the identification of two hChkl cDNAs. Neither of these clones were complete, as the longest CDNA was lacking a few base pairs near the 5′ end. The full-length clone was constructed by ligating the 5′ end of the short clone onto the longer cDNA clone to produce the sequence shown in FIG. 1 (SEQ ID NO: 1).

Once the hinnan homolog was identified, human Chkl cDNA was used to isolate the murine Chkl (mChkl). A mouse T cell CDNA library (ATCC Accession No. ATC 87291) was screened using a NotI-ClaI fragment from the hChkl cDNA as a probe as described above for the human library screen, with the exception being that the high stringency wash was conducted at 42° C. In addition, a genomic clone was isolated from a mouse ES cell library that contains chkl. This clone was found to contain the chkl exons (i.e., the sequence provided in SEQ ID NO: 2).

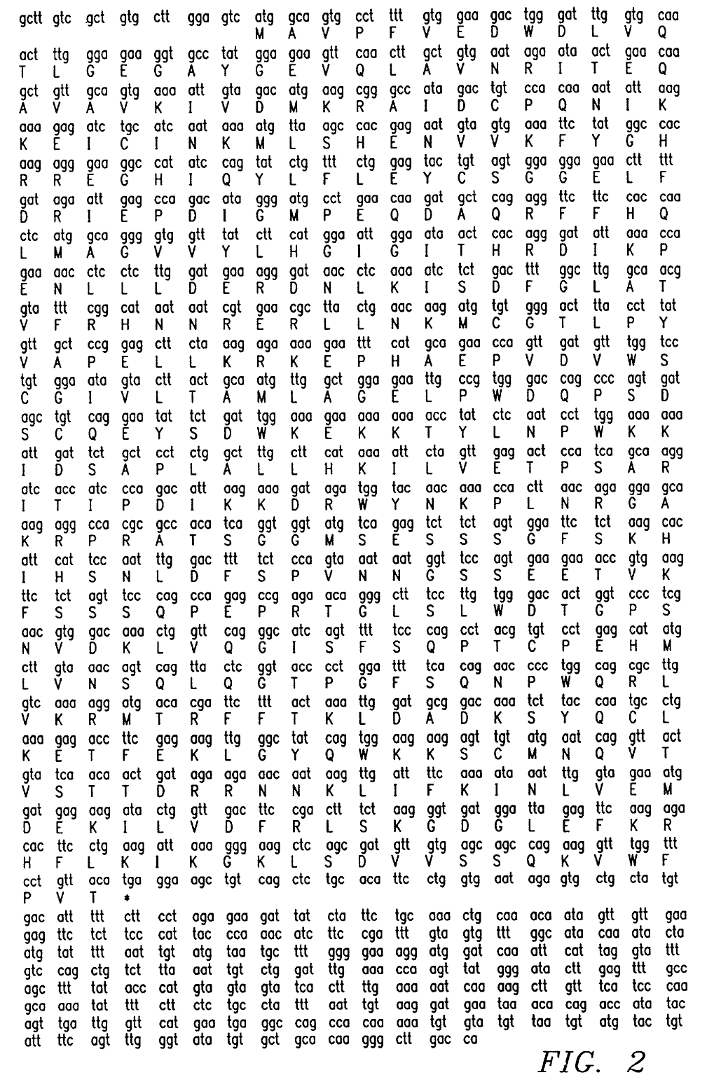

The sequence of the longest human cDNA (1891 base pairs) predicted a translation product of 476 amino acids, with an approximate size of 54 kD. No in-frame stop codon was found upstream of the first methionine, which is located with the Kozak consensus sequence (See, Kozak, Cell 44:283 [1986]), and is likely to be the initiation codon, as its encoded protein is the same size as that observed in cells (as discussed below). FIG. 1 shows the sequence of the cDNA encoding human chkl (SEQ ID NO: 1)(Genbank Accession No. AF016582), as well as the predicted amino acid sequence for the human Chkl protein (SEQ ID NO: 3). FIG. 2 shows the cDNA sequence of murine chkl (SEQ ID NO: 2)(Genbank Accession No. AF016583), as well as the predicted amino acid sequence for the murine Chkl protein (SEQ ID NO: 4).

The human Chkl gene is likely to be related to a Caenorabditis elegans gene and the Drosophila melanogaster gene grp. However, during this experimental work, it was determined that the database DNA sequence for ceChkl has a likely frame shift in the COOH-terminus. FIG. 3 shows the domain structure of the predicted human Chkl (hChkl) protein. In this Figure, the black boxes indicate regions of highest conservation. FIG. 4 shows the alignment of Chkl homologs. In this Figure, amino acid identities are shown as black boxes, and conservative changes are shown as shaded boxes. In this Figure, “Hs” indicates Homo sapiens, “Sp” indicates S. pombe, “Ce” indicates C. elegans, and “Dm” indicates D. melanogaster. In this Figure, the human sequence is SEQ ID NO: 7, the D. melanogaster sequence is SEQ ID NO: 8, the C elegans sequence is SEQ ID NO: 9, and the S. pombe sequence is SEQ ID NO: 10.

The predicted hChkl protein was found to be 29% identical and 44% similar to spChkl, 40% identical and 56% similar to the ceChkl, and 44% identical and 56% similar to Dmchkl. Sequence analysis revealed several COOH-terminal domains that appear to be highly conserved in the Chkl family of kinases.

### EXAMPLE 2

### Mapping of Chkl and its Expression

In this Example, Northern analysis of hChkl and murine hChkl was used to identify tissues that express Chkl. In addition, the chromosomal location of Chkl was mapped to 11 q24, by fluorescence in situ hybridization (FISH), as known in the art.

In the in situ hybridization experiments, tissues from adult mice, and murine embryos from day 15.5 post coitum (p.c.) were examined. Embryos or tissues were collected and fixed in 4% paraformaldehyde, embedded in paraffin and sectioned on a microtome (Zeiss) at 5 μ. Specimens were hybridized with cc-35S-UTP labelled riboprobes essentially as described (O. H. Sudin et al., Develop., 108:47; and B. Lutz et al., Develop., 120:25 [19941). Briefly, pBluescript-Chkl was linearized using either BstEl and sense and antisense transcripts were generated using either T7 or T3 polymerase, respectively. Specimens were photographed by double exposure using darkfield illumination with a red filter and Hoechst epifluorescence optics.

In addition to the in situ hybridization with murine tissues, FISH hybridization was used to map the genomic fragment containing hchkl. For this analysis, metaphase chromosomes prepared from human cells (peripheral blood lymphocytes) were tested with fluorescently-labelled human chkl DNA, as known in the art (See e.g., J. W. Ijdo et al., Genomics 14:1019-1025 [1992]). Briefly, the longest cDNA obtained from the human B cell library (See, Example 1) was subcloned into pBlueScript (Stratagene), and used as a probe to screen a human genomic library in the BAC vector by hybridization as described by the manufacturer (Genome Systems). One clone designated “BACH-190 (CI6)” (Genome Systems control number 12883) was analyzed by PCR. The PCR conditions were the same as those described in Example 1 above, with the exceptions being that a 42° C. annealing temperature was used, and two primer pairs (primers 186 and 484; and 415 and 177) were used. The sequences of the primers are shown below:

Primer 177: 5′-cta gag gag cag aat cg-3′ (SEQ ID NO: 12)

| | | |
|---|---|---|
| Primer 186: | 5′-gca gtt tgc agg aca gga taa tct tct cta gga ag-3′ | (SEQ ID NO: 13) |
| Primer 415: | 5′-ttg ctc cag aac ttc tg-3′ | (SEQ ID NO: 14) |
| Primer 484: | 5′-tat tgg ttg act tcc ggc-3′ | (SEQ ID NO: 15) |

By automated sequence analysis, it was determined that this clone contained the chkl sequence. This clone was then used in FISH analysis as known in the art, in order to determine the chromosomal location of the chkl gene.

The results of this analysis placed the gene at a position that is adjacent to the gene encoding ATM on chromosome II (i.e., at 11 q23). Loss of heterozygosity at this region has been associated with a number of cancers, including breast, lung, and ovarian cancers (I. Vorechovsky et al., Cancer Res., 56:2726 [1996]; and H. Gabra et al., Cancer Res., 56:950 [1996]).

In the Northern analyses, mRNAs from human and mouse tissues were hybridized with 25 ng of labeled human or mouse cDNAs, as appropriate, overnight in 50 mM PIPES, 100 mM NaCl, 50 mM Na₂HPO₄, 1 mM EDTA, and 5% SDS, at 65° C. The blots were washed in PIPES at room temperature, followed by a high stringency wash in 0.1×SSC with 0.5% SDS, at 65° C., for 40 minutes.

The results of the Northern blot analysis (as shown in FIGS. 5A and 5B), revealed the ubiquitous expression of hChkl, with large amounts present in human thymus, testis, small intestine, and colon. In adult mice, mChkl was detected in all tissues examined, and large amounts were found in the testis, spleen, and lung. In addition, mouse embryos from embryonic day 15.5 also revealed ubiquitous expression, with large amounts detected in the brain, liver, kidney, pancreas, intestines, thymus, and lung. These results were of particular interest, as testis, spleen, and thymus have also been found to express large amounts of ATM (G. Chen and E. Y. H. P. Lee, J. Biol. Chem., 25 271:33693 [1996]; and N. D. Lakin et al., Oncogene 13:2707 [1996]).

### EXAMPLE3

### Antibodies Against Chkl

In this Example, affinity-purified antibodies to hchkl protein (“anti-FL”) and the 15 amino acids present on the carboxy terminus of the hChki protein (“anti-PEP”) were produced. In these experiments, hChkl protein was first produced in baculovirus as described below.

Recombinant baculovirus encoding glutathione S-transferase (GST) fusion proteins to hCHkl (GST-hChkl) or a to a mutation of hChkl in which Asp at position 130 was mutated to Ala (GST-hChkl (D130A) were produced. Recombinant baculovirus encoding GST-hChkl and GST-hChkl(D130A) (pYS71) were made by introducing an Ndel at the first ATG of the hChkl open reading frame (ORF) using PCR, and subcloning the hChkl cDNA as an Nde I-EcoRI fragment into pGEX2Tcs (Invitrogen) to generate pYS45. The XbaI-EcoRl fragment from pYS45 containing GST-hChkl was then subcloned into pVL1393 (Invitrogen), which was cut with XbaI-EcoRI to generate pYS63.

The GST-hChkl(D130A) mutant was generated by the PCR and the XhoI-XmnI fragment containing the mutation was used to replace the wild-type fragment to generate pYS64. The hGST-Chkl(D130A) fragment from pYS64 was then subcloned into the baculovirus transfer vector using the Univector plasmid fusion strategy, as described in co-pending U.S. patent application Ser. No. 08/864,224, hereby incorporated by reference.

Viruses were generated by standard methods (e.g., Baculogold, Pharmingen). Recombinant GST-hChkl protein was isolated from infected HiS insect cells (Invitrogen) on glutathione (GSH) agarose (Pharmacia).

The GST-hChkl protein was then used to produce affinity-purified antibodies. In addition, antibodies directed against the carboxy-tenninal 15 amino acids were produced using synthetically produced sequence. Recombinant GST-hChkl was affinity purified from the cell lysate by chromatography on Glutathione Sepharose 4B (Pharmacia) according to the manufacturer's instructions.

Polyclonal antibodies against the purified GST-hChkl or the carboxy terminal amino acids were generated in New Zealand white rabbits (Bethyl Laboratories), using standard techniques (See e.g., E. Harlow and D. Lane, Antibodies: A Laboratory Manual, Cold Spring Harbor Press, New York [1988]). Briefly, rabbits were given an initial immunization comprising 100 μg of affinity purified GST-hChkl or the 15 amino acid sequence in complete Freund's adjuvant (CFA). The antigen was delivered by SC injection. The animals received boosts comprising 50 pg of affinity purified GST-hChkl or 15 amino acid sequence, in incomplete Freund's adjuvant (IFA), as appropriate, at the following intervals day 14, day 28, day 42, day 56, and day 70. Sera were collected by bleeding the rabbits from the ear vein and the sera were prepared using standard techniques (E. Harlow and D. Lane, supra, at pp. 117 and 119). The anti-hCHkl antibodies were referred to as “anti-FL,” while the antibodies directed against the carboxy-terminal 15 amino acids were referred to as “anti-PEP.”

Anti-PEP antibodies were purified using an affinity column that was prepared by coupling a peptide representing the carboxy-terminal 15 amino acids, at its N terminus to activated CH-Sepharose (Pharmacia) according to the manufacturer's instructions. The anti-FL antibodies were purified using an affinity column that was prepared by coupling the GST-Chkl fusion protein from baculovirus to Affi-Gel 10 (Biorad) according to the manufacturer's directions. The antibody concentrations were roughly determined by Bradford analyses. The antibodies were subsequently tested in titration experiments and in Western blots, to determine their titer and specificity.

Affinity purified antibodies to these hchkl protein made in baculovirus (“anti-FL”) or to its COOH-terminal 15 amino acids (“anti-PEP”), recognized a 54-kD protein (FIG. 6) that comigrates with hChkl expressed in baculovirus. The anti-PEP but not anti-FL signal is competed by addition of excess peptide indicating that the two sera are recognizing different hChkl epitopes, further confirming identity of the 54-kD band as endogenous hChkl. A 70-kD protein was also specifically recognized by anti-PEP.

Antibodies directed against mChkl were also produced and purified, using the same methods as described above for the anti-hChkl antibodies.

mChkl expressed from the cytomegalovirus promoter, CMV, in baby hamster kidney cells (BHK) resulted in detection of a 54-kD nuclear protein only in transfected cells using antibodies directed against the C-terminal peptide of mChkl. These (and all other transfections) were carried out as follows. Tissue culture flasks (T25) at 70-80% confluence were incubated with 3-9 μg DNA and 15-18 μl lipofectamine (Gibco BRL), in 3 ml of OptiMEMI (Gibco BRL), for 5-7 hours at 37° C. The cells were washed three times with Dulbecco's PBS without calcium or magnesium, and fed with DMEM with high glucose (Gibco BRL) and 10% FBS (Gibco BRL). The cells were harvested for Western blots or FACS analyses 48 hours post transfection. The results indicated exogenous mChkl comigrates with endogenous mchkl from mouse lung tissue.

### EXAMPLE4

### Effect of DNA Damage

To determine whether hChkl is modified in response to DNA damage like spChkl, hChkl protein in extracts from cells treated with ionizing radiation was examined.

In the first set of experiments, HeLa cells were synchronized with 2 mM thymidine, and treated without (−) or with (+) 10 Gy of ionizing radiation one hour after release from the block. Cells were collected in G2-M, and extracts were fractionated by 10% SDS-PAGE, and immunoblotted with anti-PEP.

In addition to the HeLa cells, Jurkat cells were treated (+IR) or not treated (−IR) with 10 Gy of ionizing radiation and incubated for two hours. Extracts from these cells were resolved in the first dimension by using isoelectric focusing (IEF), with pH 3 to 10 ampholytes, and in the second dimension on a 10% SDS-PAGE, followed by immunoblotting with anti-PEP.

hChkl from extracts from damaged cells showed a minor but reproducible reduction in mobility compared to Chkl^{Hs} from untreated cells (FIG. 7). This modification was confirmed by 2-dimensional gel analysis which clearly demonstrated the generation of a more negatively charged Chkl species 2 hours after ã-irradiation (FIG. 7). These results indicate that hChkl may participate in transduction of the DNA damage signal like spChkl.

Indirect immunofluorescence was also conducted. In these experiments, human fibroblasts were fixed, stained with 4′6′-diamidino-2-phenylindole (DAP) to detect DNA, and were probed with affinity-purified anti-PEP, biotinylated antibody to rabbit IgG, and Texas Red streptavidin to reveal the subcellular location of the hchkl protein. This indirect immunofluorescence revealed that hChkl is localized to the nucleus in a punctate staining pattern, similar to that observed for ATM. mChkl was also tested as described above for hChkl, with the exception that it was expressed in BHK cells. These results also confirmed the nuclear localization of mChkl.

Finally, in order to test for the ability of Chkl^{Hs} to regulate the cell cycle, hChkl or hChkl (D130A) were transfected under the control of the cytomegalovirus (CMV) promoter, or the CMV vector alone into HeLa cells treated with and without 6 Gy of ionizing radiation. These transfections were accomplished as described in Example 3, above. No perturbation of the cell cycle by either kinase relative to vector alone was detected, suggesting that overproduction alone was insufficient to deregulate the system.

### EXAMPLE 5

### Phosphorylation of Cdk Tyrosine Phosphorylation Regulators

In this Example, the effects of phosphorylation of key regulators of Cdk tyrosine phosphorylation by chkl was investigated.

Tyrosine phosphorylation of Cdc2 has been implicated in cell cycle arrest in response to DNA damage and replication blocks in both S. pombe (T. Enoch and P. Nurse, Cell 60:665 [1990]), and humans (P. Jin et al., J. Cell Biol., 134:963 [1996]). In S. pombe, Cdc2 mutants that cannot be phosphorylated on tyrosine display an inability to arrest the cell cycle in response to blockade of DNA replication. Although it was originally thought that the DNA damage checkpoint did not operate through tyrosine phosphorylation, tyrosine phosphorylation is apparently required for S. pombe cells to arrest in response to DNA damage. While it is now clear that tyrosine phosphorylation is required for proper checkpoint control, the experiments implicating tyrosine phosphorylation in this pathway do not distinguish between a regulatory role in which tyrosine phosphorylation rates are manipulated by the checkpoint pathways, or a passive role in which tyrosine phosphorylation is required to allow cell cycle arrest, but is not the actual target of the checkpoint pathway (S. J. Elledge, Science 274: 1664 [1996]; and D. J. Lew and S. Kombluth, Curr. Opin. Cell. Biol., 8:795 [1996]).

Next, the ability of hChkl to phosphorylate key regulators of Cdk tyrosine phosphorylation, the Cdc25 dual specificity phosphatases, hCdc25A, hCdc25B, and hCdc25C was analyzed. These regulators were chosen for several reasons. First, overproduction of hCdk4 mutants in which the inhibitory tyrosine is changed to phenylalanine abrogates GI arrest in response to UV light (Y. Terada et al., Nature 376:358 [1995]). Secondly, the UV-sensitivity of chkl mutants in S. pombe is suppressed by inactivating cdc25 with a Ts mutation (N. C. Walworth et al., Nature 363:368 [1993]). Finally, in S. pombe weelmikl mutants, DNA damage still causes a partial cell cycle delay that could be due to regulation of spCdc25 activity.

GST-hChkl and GST-hChkl(D130A) were introduced into baculovirus, purified from baculovir-us-infected insect cells as described in Example 3 above, and incubated with either GST, His₆-Cdc25C, GST-hCdc25A, hGST-Cdc25B, GSThCdc25C, or GST-Cdc25C(200-256), and ã³²P) ATP.

The kinase reactions contained hGST-Chkl bound to GSH agarose and either His₆-Cdc25C, GST-Cdc25A, GST-Cdc25B, GST-Cdc25C or GST-Cdc25C(200 to 256) (i.e., amino acids 200 to 256 of Cdc25). Kinase reactions contained I to 3 μg of GST-hChkl or GST-hChkl(D130A) protein on beads and soluble substrate in 20 mM Hepes (pH 7.4), 10 mM MgCl₂, 10 mM MgCl₂, 2 iM ATP and 15 iCi (ã−³²P)ATP for 30 minutes at 30° C. The proteins were resolved by SDS-PAGE (10%), and visualized by autoradiography for kinase assays (FIG. 8A), or by Coomassie staining (FIG. 8B). Less GST-Cdc25B was loaded than the other substrates (approximately ⅕ of the other substrates was loaded).

GST-Chkl phosphorylated all three Cdc25 proteins but not GST alone (FIG. 8). Although Gst-Cdc25C^{Hs} co-migrated with Gst-Chkl^{Hs} which autophosphorylates, increased phosphorylation was observed at that position relative to that in the presence of kinase alone and phosphorylation of a Gst-Cdc25C^{Hs} breakdown product was visible.

Protein kinases often form complexes with their substrates. To examine this for hChkl, and the Cdc25 proteins, GST-Cdc25 proteins present on glutathione beads were incubated together with baculovirus extracts expressing His₆-tagged hChkl, and precipitated. GST-hCdc25A, GST-hCdc25B, and GST-hCdc25C each specifically bound hChkl while GST alone did not (FIG. 10). Furthermore, two other GST fusion proteins, GST-Dunl and GST-Skpl, all failed to bind hchkl. These results indicate that Cdc25 can form complexes with hChkl.

To determine the site on Cdc25C that is phosphorylated by hChkl, the kinase reactions were carried out in a buffer consisting of 50 mM Tris (pH 7.4), 10 mM MgCl₂ 10 iM ATP, 1 mM DTT and 10 iCi (ã−³²P)ATP. The proteins were separated by SDS-PAGE, transferred to nitrocellulose membranes, and visualized by autoradiography. The nitrocellulose membrane containing His-Cdc25C was excised, blocked with 0.5% polyvinylpyrrolidone (PVP-40) in 100 mM acetic acid for 30 minutes at 37° C., washed six times with water, and digested with TPCK trypsin (Worthington) at a final concentration of 30 mg/ml, in 0.1 M NH₄CO₃ (pH 8.0). Further digestion on selected HPLC fractions was performed with 2 units of proline specific endopeptidase (ICN) in 0.1 M sodium phosphate, 5 mM EDTA (pH 7.4), at 37° C. for 16 hours. Samples were acidified in 1% trifluoroacetic acid (TFA) and loaded onto a Vydac C18 column (25 cm×0.46 cm inner diameter). Reverse phase HPLC was performed at 37° C. Reactions were loaded in 0.1% TFA (Buffer A) and eluted with a gradient from 0 to 60% Buffer B (90% acetonitrile, 0.095% TFA). Fractions were collected at 0.5 minutes intervals up to 90 minutes, and counted for radioactivity. Selected fractions were immobilized on Sequenlon-AA membrane discs (Millipore) for NH₂-terminal sequencino. Manual Edman degradation was done as known in the art (See, J. E. Bodwell et al., J. Biol. Chem., 266:7549 [1991]; and S. Sullivan, and T. W. Wong, Anal. Biochem., 197: 65 [1991]) with a coupling and cleavage temperature of 55° C.

To establish the significance of the Cdc25 phosphorylation, the site of Chkl^{Hs} phosphorylation on Cdc25C was mapped. Ser 216 is the main site of phosphorylation of Cdc25C^{Hs} in vivo. hChkl phosphorylated a 56 amino acid region of the hCdc25C protein fused to GST, but not GST alone (FIG. 8). This 56 amino acid motif contains 4 possible sites of phosphorylation. Peptide analysis of proteolytic fragments of full-length His₆-hCdc25C phosphorylated with GST-hChkl revealed a single phosphorylated tryptic peptide by high-pressure liquid chromatography. Edman degradation of this peptide indicated release of radioactivity in the third cycle (FIG. 12B). FIG. 12A shows the radioactivity measured from column fractions obtained during reverse phase HPLC. Further degradation of this tryptic fragment with proline endopeptidase resulted in a peptide that released radioactivity in the first cycle. Serine 216 is the only site on Cdc25C^{Hs} consistent with this phosphorylation pattern (FIG. 11), as amino acids inclusive of and surrounding Serine²¹⁶ contain amino-terminal trypsin and proline endopeptidase cleavage sites.

In addition, GST-hChkl purified from baculovirus was incubated with either GST-hCdc25C(200-256) or GST-hCdc25C(200-256)(S216A), and (ã−³²P)ATP, using the sane methods as described above. The results are shown in FIG. 9A). In addition, hChkl-His₆ purified from baculovirus was incubated with either GST-hCdc25C (lane 5, FIG. 9B), or GST-hCdc25C(S216A) and (ã−³²P)ATP. Proteins were resolved and visualized as described above. As shown in FIG. 9, there was clear phosphorylation of GST-hCdc25C. A catalytically inactive mutant (GSThChkl(D130A)(k-) failed to phosphorylate itself or any of the Cdc25 proteins (See, FIG. 9).

To confirm this, the Cdc25C S216A mutation in Gst-Cdc25C and Cdc25C(200-256) were constructed. Both were found to be poor substrates for hChkl confirming S216 as the site phosphorylation (FIG. 11). S216 has also been reported to be phosphorylated by spChkl, demonstrating phylogenetic conservation of this regulatory relationship.

### EXAMPLE 6

### Production of Monoclonal Antibodies

The antibodies of the present invention may be monoclonal or polyclonal. Thus, it is within the scope of this invention to include other (e.g., second antibodies) (monoclonal or polyclonal) directed against or similar to the first antibodies discussed above. It is contemplated that these antibodies will find use in detection assays. Both the first and second antibodies may be used in the detection assays or a first antibody may be used with a commercially available anti-immunoglobulin antibody. An antibody as contemplated herein includes any antibody specific to any region of human or murine Chkl.

The production and use of monoclonal antibodies in an immunoassay is an alternative method to that described in Example 3. Monoclonals provide some advantages because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art. (See e.g., Douillard and Hoffman, Basic Facts about Hybridomas, in Compendium of immunology Vol II, ed. by Schwartz [1981]; Kohler and Milstein, Nature 256: 495-499 [1975]; Eur. J. Immunol., 6: 51 I-519, [19761).

Unlike preparation of polyclonal sera, the choice of animal is dependent on the availability of appropriate immortal lines capable of fusing with lymphocytes. Mouse and rat have been the animals of choice in hybridoma technology and are preferably used. Humans can also be utilized as sources for sensitized lymphocytes if appropriate inu-nortalized human (or nonhuman) cell lines are available. For the purpose of the present invention, the animal of choice may be injected with an antigenic amount, for example, from about 0.1 mg to about 20 mg of the enzyme or protein or antigenic parts thereof. Usually the injecting material is emulsified in Freund's complete adjuvant. Boosting injections may also be required. The detection of antibody production can be carried out by testing the antisera with appropriately labelled antigen. Lymphocytes can be obtained by removing the spleen of lymph nodes of sensitized animals in a sterile fashion and carrying out fusion. Alternatively, lymphocytes can be stimulated or immunized in vitro, as described, for example, in Reading, J. Immunol. Meth., 53: 261-291 [1982]).

A number of cell lines suitable for fusion have been developed and the choice of any particular line for hybridization protocols is directed by any one of a number of criteria such as speed, uniformity of growth characteristics, deficiency of its metabolism for a component of the growth medium, and potential for good fusion frequency.

Intraspecies hybrids, particularly between like strains, work better than interspecies fusions. Several cell lines are available, including mutants selected for the loss of ability to secrete myeloma immunoglobulin.

Cell fusion can be induced either by virus, such as Epstein-Barr or Sendai virus, or polyethylene glycol. Polyethylene glycol (PEG) is the most efficacious agent for the fusion of mammalian somatic cells. PEG itself may be toxic for cells and various concentrations should be tested for effects on viability before attempting fusion. The molecular weight range of PEG may be varied from 1000 to 6000. It gives best results when diluted to from about 20% to about 70% (w/w) in saline or serum-free medium. Exposure to PEG at 37° C. for about 30 seconds is preferred in the present case, utilizing murine cells. Extremes of temperature (i.e., about 45° C.) are avoided, and preincubation of each component of the fusion system at 37° C. prior to fusion can be useful. The ratio between lymphocytes and malignant cells is optimized to avoid cell fusion among spleen cells and a range of from about 1:1 to about 1:10 is commonly used.

The successfully fused cells can be separated from the myeloma line by any technique known by the art. The most common and preferred method is to choose a malignant line which is Hypoxthanine Guanine Phosphoribosyl Transferase (HGPRT) deficient, which will not grow in an aminopterin-containing medium used to allow only growth of hybrids and which is generally composed of hypoxthanine 1.10-⁴M, aiminopterin 1×10⁻⁵M, and thymidine 3×10⁻⁵M, commonly known as the HAT medium. The fusion mixture can be grown in the HAT-containing culture medium immediately after the fusion 24 hours later. The feeding schedules usually entail maintenance in HAT medium for two weeks and then feeding with either regular culture medium or hypoxthanine, thymidine-containing medium.

The growing colonies are then tested for the presence of antibodies that recognize the antigenic preparation. Detection of hybridoma antibodies can be performed using an assay where the antigen is bound to a solid support and allowed to react to hybridoma supernatants containing putative antibodies. The presence of antibodies may be detected by “sandwich” techniques using a variety of indicators. Most of the common methods are sufficiently sensitive for use in the range of antibody concentrations secreted during hybrid growth.

Cloning of hybrids can be carried out after 21-23 days of cell growth in selected medium. Cloning can be preformed by cell limiting dilution in fluid phase or by directly selecting single cells growing in semi-solid agarose. For limiting dilution, cell suspensions are diluted serially to yield a statistical probability of having only one cell per well. For the agarose technique, hybrids are seeded in a semi-solid upper layer, over a lower layer containing feeder cells. The colonies from the upper layer may be picked up and eventually transferred to wells.

Antibody-secreting hybrids can be grown in various tissue culture flasks, yielding supernatants with variable concentrations of antibodies. In order to obtain higher concentrations, hybrids may be transferred into animals to obtain inflammatory ascites. Antibody-containing ascites can be harvested 8-12 days after intraperitoneal injection. The ascites contain a higher concentration of antibodies but include both monoclonals and immunoglobulins from the inflammatory ascites. Antibody purification may then be achieved by, for example, affinity chromatography.

Antibodies produced by these methods can then be used in immunoassay methods to detect human or murine Chkl. Such methods include, but are not limited to ELISA (enzyme-linked immunosorbent assay), IFA (immunofluorescence assay), or RIA (radioimmunoassay).

From the above it should be clear that the present invention provides gene sequences encoding mammalian checkpoint genes and proteins useful as probes for tumors and other malignancies, as well as growth and/or development deficiencies.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

What is claimed is:

1. An antibody, wherein said antibody is capable of specifically binding to at least one antigenic determinant on the proteins encoded by an amino acid sequence selected from the group comprising SEQ ID NOS: 3, 4, 7, 8, 9, and 10.

2. The antibody of claim 1, wherein said antibody is a polyclonal antibody.

3. The antibody of claim 1, wherein said antibody is a monoclonal antibody.

4. A method for producing anti-Chkl antibodies comprising:
a) providing in any order:
i) an antigen comprising at least a portion of Chkl protein;
ii) an animal having immunocompetent cells; and
b) exposing said animal to said Chkl protein under conditions such that said immunocompetent cells produce anti-Chkl antibodies.

5. The method of claim 4, further comprising the step of harvesting said antibodies.

6. The method of claim 4, wherein said antigen comprising at least a portion of Chkl protein is a fusion protein.

7. The method of claim 4, further comprising the step of fusing said immunocompetent cells with an immortal cell line under conditions such that a hybridoma is produced.
